(19) United States
(12) Patent Application Publication
(10) Pub. No.: US 2023/0383265 A1
(43) Pub. Date: Nov. 30, 2023

(54) D-beta-HYDROXYBUTYRATE DEHYDROGENASES (BHBDhs) AND THEIR USE FOR KETONE MONITORING

(71) Applicants: The University of North Carolina at Chapel Hill, Chapel Hill, NC (US); National University Corporation Tokyo University of Agriculture and Technology, Tokyo (JP)

(72) Inventors: Koji SODE, Chapel Hill, NC (US); Junko SHIMAZAKI, Chapel Hill, NC (US); Wakako TSUGAWA, Tokyo (JP)

(21) Appl. No.: 18/322,823

(22) Filed: May 24, 2023

Related U.S. Application Data
(60) Provisional application No. 63/365,252, filed on May 24, 2022.

(51) Int. Cl. C12N 9/04; C12Q 1/00

(52) U.S. Cl. CPC C12N 9/0006; C12Y 101/0103; C12Q 1/005

(57) ABSTRACT

Engineered D-β-hydroxybutyrate dehydrogenases (BHBDhs) are disclosed. In some embodiments, the BHBDhs are derived from enzymes which are putative homologues of A. faecalis-derived BHBDh, including from thermophilic bacteria and radiation-resistance bacteria. These BHBDhs may also comprise additional mutations to increase their catalytic activity. Further, BHBDhs modified to include an NAD-binding loop, so as to tightly bind an NAD⁺ co-factor, are also disclosed.

### CROSS-REFERENCE TO RELATED APPLICATION

This application claims the benefit of and priority to U.S. Provisional Application No. 63/365,252, filed on May 24, 2022, which is incorporated by reference herein in its entirety.

### SEQUENCE LISTING

The instant application contains a Sequence Listing which has been submitted electronically in XML format and is hereby incorporated by reference in its entirety. Said XML copy, created on Jul. 6, 2023, is named 596756subseqlist.xml and is 22.3 kilobytes in size.

### BACKGROUND

Diabetic ketoacidosis (DKA) is a life-threatening complication which is characteristic in individuals with insulin deficiency. It occurs predominantly in Type 1 diabetic patients, but is also observed in Type 2 diabetes or gestational diabetes patients. DKA is resulted from the shortage of insulin and increased regulatory hormones like glucagon and cortisol, which leads to hyperglycemia and accumulation of ketones in blood [Kitabchi (2009); Dhatariya (2016); Dhatariya (2020); Diabetes.co.uk (2019)]. This leads to low glucose uptake and fatty acids are utilized as energy source instead of carbohydrates which can cause ketosis. In normal metabolism, fatty acids are oxidized to produce acetyl CoA, which is utilized in TCA cycle.

In insulin deficiency, excess acetyl CoA is produced and converted into ketone bodies; R-3-hydroxybutyrate (PubChem CID 92135, major synonyms are D-3-hydroxybutyrate, D-β-hydroxybutyrate, 3-D-hydroxybutyrate, abbreviated BHB in this specification), acetoacetate which is then broken down into acetone. The American Diabetes Association advises that blood ketone testing methods to quantify BHB would be desirable for diagnosis and monitoring of ketoacidosis. Ketoacidosis occurs when ketone bodies are produced faster than metabolic breakdown [Mitchell (1995); Laffel (1999)]. Euglycemic DKA has been identified as a potential morbidity in diabetic patients who take sodium-glucose co-transporter 2 (SGLT-2) inhibitor [Peters (2016); FDA (2015); EMA (2016)]. SGLT-2 inhibitors block SGLT-2, which absorbs glucose from urine and returns it to the bloodstream as the blood is filtered in the kidneys. By blocking SGLT-2, these medicines cause more glucose to be eliminated in the urine, lowering the blood glucose level with an insulin independent mechanism. Patients treated with SGLT-2 inhibitors may be at higher risk for unrecognized DKA due to increased urinary glucose excretion (in the range of 50 to 100 g/day), resulting in lower blood glucose levels than would be expected in the onset of DKA [Rosenstock (2015)]. In clinical practice, rare but serious DKA has been reported in type 2 diabetes patients treated with SGLT-2 inhibitors and more commonly (absolute risk increase ˜10% per year) in type 1 diabetes. Thus, ketone sensing technologies are receiving greater attention to prevent or improve outcomes of DKA.

In DKA, BHB is the predominant metabolite (BHB 78%, acetoacetate 20%, acetone 2%) [Wallace (2004)]. At present, the measurement of ketone bodies is performed using blood or urine in the sample. For the measurement of BHB, blood or interstitial fluid (ISF) are considered better testing matrices, because concentration in urine does not equate to the plasma ketone concentration [Dhatariya (2016)].

A commonly used rule for interpreting blood ketone levels is that normal levels of BHB can be defined as <0.6 mM, ketosis can be defined as levels of 0.6-1.5 mM, hyperketonemia can be defined as levels of 1.5-3.0 mM, and ketoacidosis can be defined as levels in excess of 3.0 mM [Diabetes.co.uk (2019)]. However, current commercially available disposable single use sensor strips for blood ketone levels can only offer a snapshot that reflects ketonemia during a specific point in time. Precise dynamic information of blood ketone concentration and, “ketone variability” will be expected to manage and prevent DKA in the diabetic patients [Pluddemann (2011)], which will be achieved by the development of continuous ketone monitoring system.

Further, present hand-held point-of-care tests (POCT) or personal use sensors for the measurement of BHB are constructed similar to blood glucose monitors (BGM) and are based on an oxidoreductase to catalyze the oxidation of BHB. However, the oxidoreductases can suffer from stability issues, as well as the need to have a cofactor present.

It is in an effort to address the above limitations that the following invention has been developed.

### BRIEF SUMMARY

An embodiment is an engineered β-hydroxybutyrate dehydrogenase (BHBDh) with increased thermal stability as compared to the wild-type.

An embodiment is an engineered β-hydroxybutyrate dehydrogenase (BHBDh) having a NAD-binding sequence inserted therein.

An embodiment is an expression vector comprising a nucleic acid sequence encoding any engineered BHBDh described herein.

An embodiment is a method of producing any engineered BHBDh described herein, the method comprising the steps of
i) culturing a host cell transfected with a vector comprising a nucleic acid sequence encoding the engineered BHBDh under conditions suitable for expression of the polypeptide from the host cell, and
ii) recovering the engineered BHBDh.

An embodiment is a method of assaying β-hydroxybutyrate (BHB) in a sample, the method comprising the steps of
contacting the sample with any engineered BHBDh described herein; and
measuring an amount of the BHB oxidized by the engineered BHBDh.

An embodiment is a device for assaying BHB in a sample, the device comprising: any engineered BHBDh described herein; and an electron mediator.

An embodiment is a kit for assaying BHB in a sample, the kit comprising: any engineered BHBDh described herein; and an electron mediator.

An embodiment is an enzyme electrode comprising any engineered BHBDh described herein immobilized on an electrode.

### BRIEF DESCRIPTION OF THE SEVERAL VIEWS OF THE DRAWING(S)

Having thus described the invention in general terms, reference will now be made to the accompanying drawings, which are not necessarily drawn to scale.

FIG. 1 illustrates the oxidation reaction catalyzed by (R)-3-hydroxybutyrate dehydrogenase (BHBDh).

FIGS. 2-3 illustrate the stability of BHBDh derived from microorganisms (Toyobo enzyme) in powder form and solution form (incubated for 15 minutes at each temperature, with 50 mM K-phosphate buffer, at pH 6.5, with an enzyme concentration of 50 U/ml), respectively.

FIG. 4 illustrates putative amino acid sequences of BHBDhs derived from certain organisms, and the alignment of these sequences.

FIG. 5 illustrates the vector plasmids for the expression of putative BHBDhs derived from Thermus thermophiles (TtBHBDh), Geobacillus stearothermophilus (GsBHBDh) and Deinococcus radiotolerans (DrBHBDh) (where Xx at the beginning of each abbreviation represents the name of the organism from which the BHBDh is derived).

FIG. 6A illustrates the purification of putative BHBDh derived from Thermus thermophilus (TtBHBDh). FIG. 6B illustrates SDS PAGE analyses of the putative TtBHBDH, before (left) and after (right) purification.

FIG. 7A illustrates the purification of putative BHBDh derived from Geobacillus stearothermophilus (GsBHBDh). FIG. 7B illustrates SDS PAGE analyses of the putative GsBHBDH, before (left) and after (right) purification.

FIG. 8A illustrates the purification of putative BHBDh derived from Deinococcus radiotolerans (DrBHBDh). FIG. 8B SDS PAGE analyses of the putative DrBHBDH, before (left) and after (right) purification.

FIG. 9 illustrates BHB concentration dependent BHBDh activity of recombinantly prepared BHBDhs derived from Thermus thermophilus (TtBHBDh).

FIGS. 10A and 10B illustrate the thermal stability of BHBDh derived from Alcaligenes faecalis (AfBHBDh), using specific activity and relative residual activity, respectively.

FIGS. 11A and 11B illustrate results of the same thermal stability testing on TtBHBDh.

FIGS. 12A and 12B illustrate results of the same thermal stability testing on GsBHBDh.

FIGS. 13A and 13B illustrate results of the same thermal stability testing on DrBHBDh.

FIG. 14A illustrates compiled specific activity data for AfBHBDh, TtBHBDh, GsBHBDh, and DrBHBDr, in the presence of 10 mM MgCl₂. FIG. 14B illustrates compiled relative residual activity data for AfBHBDh, TtBHBDh, GsBHBDh, and DrBHBDr, in the absence of MgCl₂.

FIGS. 15A and 15B illustrate purification and SDS PAGE analysis results of a TtBHBDh Ala175Cys (CPAY) mutant, respectively.

FIGS. 16A and 16B illustrate purification and SDS PAGE analysis results of a TtBHBDh Ala177Gly (APGY) mutant, respectively.

FIGS. 17A and 17B illustrate purification and SDS PAGE analysis results of a TtBHBDh Tyr178Trp (APAW) mutant, respectively.

FIGS. 18A and 18B illustrate purification and SDS PAGE analysis results of a TtBHBDh Ala175Cys/Pro176/Ala177Gly/Tyr178Trp (CPGW) mutant, respectively.

FIG. 19 illustrates the enzyme activities of wild type and mutant TtBHBDhs; Ala175Cys (CPAY), Ala177Gly (APGY), Tyr178Trp (APAW), and Ala175Cys/Pro176/Ala177Gly/Tyr178Trp (CPGW).

FIG. 20 illustrates the thermal stabilities of wild type and mutant TtBHBDhs; Ala175Cys (CPAY), Ala177Gly (APGY), Tyr178Trp (APAW), and Ala175Cys/Pro176/Ala177Gly/Tyr178Trp (CPGW).

FIG. 21 illustrates the amino acid sequence alignments of BHBDh derived from Alcaligenes faecalis (AfBHBDh), Thermus thermophilus (TtBHBDh), Geobacillys stearothermophilus (GsBHBDh) and from Deinococcus radiotolerans (DrBHBDh).

FIG. 22 illustrates the expression vector (above) inserted by a chimeric enzyme composed of AfBHBDh inserted by a loop region (12 amino acid residues) sequence derived from a nicotinoprotein dehydrogenase, carveol dehydrogenase derived from Mycobacterium tuberculosis, (AfBHBDh 36-48_{Cd}) and its amino acid sequence (below).

FIG. 23 illustrates the expression vector (above) inserted by a chimeric enzyme composed of AfBHBDh inserted by a loop region (12 amino acid residues) sequence with two amino acid substitutions; Ser14Arg/Ser71Arg, derived from a nicotinoprotein dehydrogenase, carveol dehydrogenase derived from Mycobacterium tuberculosis (AfBHBDh 36-48_{Cd} S14R/S77R) and its amino acid sequence (below).

FIGS. 24A and 24A illustrate purification and SDS PAGE analysis results of AfBHBDh Ser14Arg/Ser71Arg mutants, respectively.

FIG. 25 illustrates the NAD dependent BHB dehydrogenase activities of AfBHBDh, and two chimeric enzymes, AfBHBDh 36-48_{Cd} and AfBHBDh 36-48_{Cd} S14R/S77R.

FIG. 26 illustrates the NADH dependent fluorescence observed from AfBHBDh, and two chimeric enzymes, AfBHBDh 36-48_{Cd} and AfBHBDh 36-48_{Cd} S14R/S77R.

FIG. 27 illustrates the dye-mediated dependent BHB dehydrogenase activities of AfBHBDh, and two chimeric enzymes, AfBHBDh 36-48_{Cd} and AfBHBDh 36-48_{Cd} S14R/S77R.

FIGS. 28A and 28B illustrate putative amino acid sequences of BHBDhs derived from organisms in addition to those already illustrated in FIG. 4, the alignment of these sequences, and additional structural information regarding those sequences.

FIG. 29 illustrates the active site of AfHBDh in conjunction with βHB, along with the alignment of a segment of the sequences depicted in FIGS. 28A and 28B. FIG. 30 is another illustration of the alignment of a segment of the sequences depicted in FIGS. 28A and 28B.

FIG. 31A depicts affinity chromatography protocol and recovery of a GsBHBDh Y185W mutant. FIG. 31B depicts SDS-PAGE of the GsBHBDh Y185W mutant before and after affinity chromatography.

FIG. 32 shows a comparison of specific activities of AfBHBDh, TtBHBDh, and GsBHBdh wild types and mutants.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

The term “subject” refers to a mammal (e.g., a human) in need of detection or quantification of an analyte, or target substance. The subject may include dogs, cats, pigs, cows, sheep, goats, horses, rats, mice, non-human mammals, and humans. The term “subject” does not necessarily exclude an individual that is healthy in all respects and does not have or show signs of elevated or lowered target substance. In embodiments, the sample is a biological sample.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers, tenths, and hundredths within or defining the range, and all subranges defined by integers within the range.

The term “in vivo” refers to natural environments (e.g., a cell or organism or body) and to processes or reactions that occur within a natural environment.

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. Examples of promoters can be found, for example, in WO 2013/176772, herein incorporated by reference in its entirety for all purposes.

“Operable linkage” or being “operably linked” includes juxtaposition of two or more components (e.g., a regulatory sequence, such as a promoter, and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof “Equivalent program” includes any sequence comparison program that, for any two sequences in question, generates an alignment having identical nucleotide or amino acid residue matches and an identical percent sequence identity when compared to the corresponding alignment generated by GAP Version 10.

Unless otherwise apparent from the context, the term “about” encompasses values within a standard margin of error of measurement (e.g., SEM) of a stated value or variations ±0.5%, 1%, 5%, or 10% from a specified value.

The singular forms of the articles “a,” “an,” and “the” include plural references unless the context clearly dictates otherwise.

Statistically significant means p≤0.05.

An embodiment is an engineered β-hydroxybutyrate dehydrogenase (BHBDh) with increased thermal stability as compared to the wild-type.

In a further embodiment, the engineered BHBDh comprises a sequence having at least 90% sequence identity to any one of SEQ ID NOs: 1-7 or 12-20, provided at least one of the amino acids at a position in said sequence corresponding to positions 165-188 is different from the amino acid occupying the corresponding position according to SEQ ID NO: 3. In a further embodiment, the engineered BHBDh comprises a sequence having at least 90% sequence identity to any one of SEQ ID NOs: 1-7, provided at least one of the amino acids at a position in said sequence corresponding to positions 165-188 is different from the amino acid occupying the corresponding position according to SEQ ID NO: 3. In an embodiment, the engineered BHBDh comprises a sequence having at least 91%, at least 92%, at least 93%, at least 94%, at least 95%, at least 96%, at least 97%, at least 98%, at least 99%, or at least 99.5% sequence identity to any one of SEQ ID NOs: 1-7 or 12-20. In an embodiment, the engineered BHBDh comprises a sequence having at least 91%, at least 92%, at least 93%, at least 94%, at least 95%, at least 96%, at least 97%, at least 98%, at least 99%, or at least 99.5% sequence identity to any one of SEQ ID NOs: 1-7.

In a further embodiment, the engineered BHBDh comprises a modification at one or more amino acid positions selected from:
(a) a position corresponding to position 175 of the amino acid sequence set forth in SEQ ID NO: 3,
(b) a position corresponding to position 177 of the amino acid sequence set forth in SEQ ID NO: 3, and
(c) a position corresponding to position 178 of the amino acid sequence set forth in SEQ ID NO: 3.

In a further embodiment, the engineered BHBDh comprises a modification at one or more amino acid positions selected from:
(a) a position corresponding to position 175 of the amino acid sequence set forth in SEQ ID NO: 3, wherein the modification includes a substitution of the wild-type amino acid residue with an amino acid residue Cys;
(b) a position corresponding to position 177 of the amino acid sequence set forth in SEQ ID NO: 3, wherein the modification includes a substitution of the wild-type amino acid residue with an amino acid residue Gly; and
(c) a position corresponding to position 178 of the amino acid sequence set forth in SEQ ID NO: 3, wherein the modification includes a substitution of the wild-type amino acid residue with an amino acid residue Trp.

In a further embodiment, the engineered BHBDh comprises a modification at:
(a) a position corresponding to position 175 of the amino acid sequence set forth in SEQ ID NO: 3, wherein the modification includes a substitution of the wild-type amino acid residue with an amino acid residue Cys;
(b) a position corresponding to position 177 of the amino acid sequence set forth in SEQ ID NO: 3, wherein the modification includes a substitution of the wild-type amino acid residue with an amino acid residue Gly; and
(c) a position corresponding to position 178 of the amino acid sequence set forth in SEQ ID NO: 3, wherein the modification includes a substitution of the wild-type amino acid residue with an amino acid residue Trp.

In a further embodiment, the engineered BHBDh comprises a sequence having at least 90% sequence identity to any one of SEQ ID NOs: 3-5. In an embodiment, the engineered BHBDh comprises a sequence having at least 91%, at least 92%, at least 93%, at least 94%, at least 95%, at least 96%, at least 97%, at least 98%, at least 99%, or at least 99.5% sequence identity to any one of SEQ ID NOs: 3-5.

In a further embodiment, the engineered BHBDh comprises a modification at one or more amino acid positions selected from:
(a) a position corresponding to position 175 of the amino acid sequence set forth in SEQ ID NO: 3, wherein the modification includes a substitution of the amino acid residue Ala with an amino acid residue Cys;
(b) a position corresponding to position 177 of the amino acid sequence set forth in SEQ ID NO: 3, wherein the modification includes a substitution of the amino acid residue Ala with an amino acid residue Gly; and
(c) a position corresponding to position 178 of the amino acid sequence set forth in SEQ ID NO: 3, wherein the modification includes a substitution of the amino acid residue Tyr with an amino acid residue Trp.

In a further embodiment, the engineered BHBDh comprises a modification at:
(a) a position corresponding to position 175 of the amino acid sequence set forth in SEQ ID NO: 3, wherein the modification includes a substitution of the amino acid residue Ala with an amino acid residue Cys;
(b) a position corresponding to position 177 of the amino acid sequence set forth in SEQ ID NO: 3, wherein the modification includes a substitution of the amino acid residue Ala with an amino acid residue Gly; and
(c) a position corresponding to position 178 of the amino acid sequence set forth in SEQ ID NO: 3, wherein the modification includes a substitution of the amino acid residue Tyr with an amino acid residue Trp.

An embodiment is an engineered β-hydroxybutyrate dehydrogenase (BHBDh) having a NAD-binding sequence inserted therein. This engineered BHBDh will be of the general structure (A)-(N)-(A′), wherein
A is an N-terminal portion of a BHBDh and A′ is a C-terminal portion of a BHBDh, wherein A and A′, together, form a complete BHBDh; and
N is an inserted sequence capable of binding NAD.

When the sequence identity of a BHBDh is referenced, this is referring to sequence identity of A and A′, together. When the sequence identity of an engineered BHBDh is referenced, this is referring to the sequence identity of the (A)-(N)-(A′) construct.

In a further embodiment, the BHBDh has a sequence having at least 90% sequence identity to any one of SEQ ID NOs: 1-7 or 12-20. In an embodiment, the BHBDh has a sequence having at least 90% sequence identity to any one of SEQ ID NOs: 1-7. In an embodiment, the BHBDh comprises a sequence having at least 91%, at least 92%, at least 93%, at least 94%, at least 95%, at least 96%, at least 97%, at least 98%, at least 99%, or at least 99% sequence identity to any one of SEQ ID NOs: 1-7 or 12-20. In an embodiment, the BHBDh comprises a sequence having at least 91%, at least 92%, at least 93%, at least 94%, at least 95%, at least 96%, at least 97%, at least 98%, at least 99%, or at least 99% sequence identity to any one of SEQ ID NOs: 1-7.

In a further embodiment, the BHBDh has a sequence having at least 90% sequence identity to SEQ ID NO: 1. In an embodiment, the BHBDh comprises a sequence having at least 91%, at least 92%, at least 93%, at least 94%, at least 95%, at least 96%, at least 97%, at least 98%, at least 99%, or at least 99% sequence identity to SEQ ID NO: 1.

In a further embodiment, at least one of the amino acids at a position in said sequence corresponding to positions 4-24, and at least one of the amino acids at a position in said sequence corresponding to positions 61-81, is different from the amino acid occupying the corresponding position according to SEQ ID NO: 1.

In a further embodiment, the BHBDh comprises a modification at one or more amino acid positions selected from:
(a) a position corresponding to position 14 of the amino acid sequence set forth in SEQ ID NO: 1; and
(b) a position corresponding to position 71 of the amino acid sequence set forth in SEQ ID NO: 1.

In a further embodiment, the chimeric BHBDh comprises a modification at one or more amino acid positions selected from:
(a) a position corresponding to position 14 of the amino acid sequence set forth in SEQ ID NO: 1, wherein the modification includes a substitution of the wild-type amino acid residue with an amino acid residue Arg; and
(b) a position corresponding to position 71 of the amino acid sequence set forth in SEQ ID NO: 1, wherein the modification includes a substitution of the wild-type amino acid residue with an amino acid residue Arg.

In a further embodiment, at least one of the amino acids at a position in said sequence corresponding to positions 165-188 is different from the amino acid occupying the corresponding position according to SEQ ID NO: 3.

In a further embodiment, the BHBDh comprises a modification at one or more amino acid positions selected from:
(a) a position corresponding to position 175 of the amino acid sequence set forth in SEQ ID NO: 3,
(b) a position corresponding to position 177 of the amino acid sequence set forth in SEQ ID NO: 3, and
(c) a position corresponding to position 178 of the amino acid sequence set forth in SEQ ID NO: 3.

In a further embodiment, the BHBDh comprises a modification at one or more amino acid positions selected from:
(a) a position corresponding to position 175 of the amino acid sequence set forth in SEQ ID NO: 3, wherein the modification includes a substitution of the wild-type amino acid residue with an amino acid residue Cys;
(b) a position corresponding to position 177 of the amino acid sequence set forth in SEQ ID NO: 3, wherein the modification includes a substitution of the wild-type amino acid residue with an amino acid residue Gly; and
(c) a position corresponding to position 178 of the amino acid sequence set forth in SEQ ID NO: 3, wherein the modification includes a substitution of the wild-type amino acid residue with an amino acid residue Trp.

In a further embodiment, the BHBDh comprises a modification at:
(a) a position corresponding to position 175 of the amino acid sequence set forth in SEQ ID NO: 3, wherein the modification includes a substitution of the wild-type amino acid residue with an amino acid residue Cys;
(b) a position corresponding to position 177 of the amino acid sequence set forth in SEQ ID NO: 3, wherein the modification includes a substitution of the wild-type amino acid residue with an amino acid residue Gly; and
(c) a position corresponding to position 178 of the amino acid sequence set forth in SEQ ID NO: 3, wherein the modification includes a substitution of the wild-type amino acid residue with an amino acid residue Trp.

In a further embodiment, the NAD-binding sequence has a length of from 8-50 amino acids, or from 10-40 amino acids, or from 10-30 amino acids, or from 10-20 amino acids.

In a further embodiment, the NAD-binding sequence is derived from a nicotinoprotein dehydrogenase.

In a further embodiment, the nicotinoprotein dehydrogenase is carveol dehydrogenase.

In a further embodiment, the carveol dehydrogenase is Mycobacterium tuberculosis carveol dehydrogenase.

In a further embodiment, the NAD-binding sequence has at least 90% sequence identity to SEQ ID NO: 10. In an embodiment, the NAD-binding sequence has at least 95% sequence identity to SEQ ID NO: 10. In an embodiment, the NAD-binding sequence is SEQ ID NO: 10.

In a further embodiment, the NAD-binding sequence is inserted after an amino acid at a position corresponding to any one of positions 15-250, or 20-200, or 25-150, or 30-100, or 35-50, or 100-240, or 150-230, or 200-220, according to SEQ ID NO: 1. In an embodiment, the NAD-binding sequence is inserted after an amino acid at a position corresponding to position 35 according to SEQ ID NO: 1.

In a further embodiment, the engineered BHBDh comprises a purification tag. In an embodiment, the purification tag is a His-tag. In an embodiment, the His-tag is a His (6×)-tag. In an embodiment, the His (6×)-tag is SEQ ID NO: 11.

In an embodiment, the purification tag is at the C-terminal end of the engineered BHBDh.

In an embodiment, the engineered BHBDh has a nicotinamide adenine dinucleotide (NAD) bound thereto.

An embodiment is an expression vector comprising a nucleic acid sequence encoding any engineered BHBDh described herein.

An embodiment is a method of producing any engineered BHBDh described herein, the method comprising the steps of
i) culturing a host cell transfected with a vector comprising a nucleic acid sequence encoding the engineered BHBDh under conditions suitable for expression of the polypeptide from the host cell, and
ii) recovering the engineered BHBDh. In an embodiment, the vector comprises the nucleic acid sequence operably linked to a promoter.

An embodiment is a method of assaying β-hydroxybutyrate (BHB) in a sample, the method comprising the steps of
contacting the sample with any engineered BHBDh described herein; and
measuring an amount of the BHB oxidized by the engineered BHBDh.

In an embodiment, the measurement is a continuous measurement.

An embodiment is a device for assaying BHB in a sample, the device comprising: any engineered BHBDh described herein; and an electron mediator.

An embodiment is a kit for assaying BHB in a sample, the kit comprising: any engineered BHBDh described herein; and an electron mediator.

An embodiment is an enzyme electrode comprising any engineered BHBDh described herein immobilized on an electrode.

An embodiment is an enzyme sensor for assaying BHB comprising the enzyme electrode as a working electrode.

### DISCUSSION AND EXAMPLES

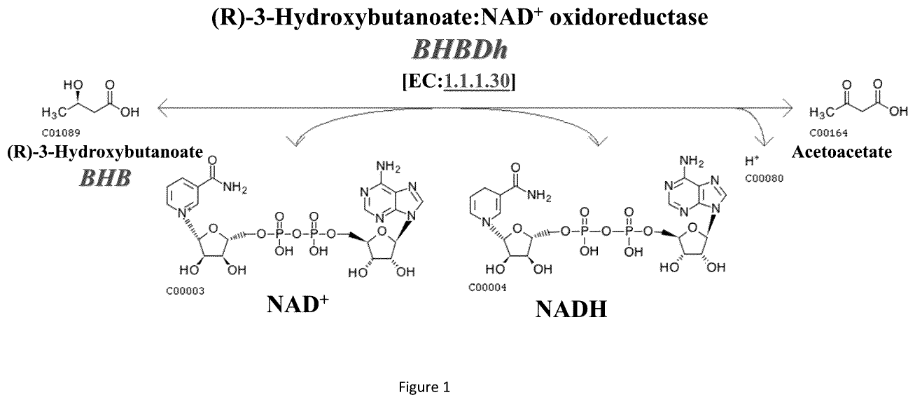

At the moment, (R)-3-hydroxybutyrate dehydrogenase (D-β-hydroxybutyrate) (BHBDh, E.C. 1.1.1.30) is the only enzyme discovered in nature which catalyzes the oxidation of BHB. BHBDh is a NAD+ dependent oxidoreductase, which catalyzes the reversible reaction between BHB and acetoacetate coupled to NAD+/NADH conversion (FIG. 1). BHB is a chiral molecule at the 3′ hydroxyl group. There are two enantiomers, R/D and S/L. BHBDhs show strict stereo-specificity to the D-enantiomer of (R)-3-hydroxybutyrate as a substrate. As a result of the chiral specificity of BHBDh, only R-3-hydroxybutyrate is produced by normal metabolism by the reduction reaction (reverse reaction of oxidation of BHB) of acetoacetate. Thus, R-3-hydroxybutyrate is the normal product of human metabolism, and is the target molecule of ketone sensing. BHBDh is found in a large variety of species from microorganisms to mammals. For diagnostic purposes, only bacteria-derived enzymes have been utilized. Bacterial BHBDhs have been recombinantly produced and used in electrochemical biosensors (Batchelor (1989); McNeil (1990); Martinez-Garcia (2017); Mascini (1988); Palleschi (1988); Marrazza (1994); Uno (1995); Forrow (2005); Li (2005); Fang (2008); Veerapandian (2016)]. BHBDhs derived from Pseudomonas species (particularly Pseudomonas lemoignei) and Arthrobacter faecalis derived BHBDhs are commercially available for diagnostic use.

BHB sensing, using BHBDhs, is based on the quantification of NADH production by the reaction of BHBDh. Thus, NAD-dependent enzyme-based sensor platforms can be applied for BHB sensing. All commercially available hand-held BHB sensors, as well as the state-of-the-art research utilize BHBDhs with hardware and test strip designs that are similar to blood glucose monitors.

Technological limitation to realize in situ, real-time continuous ketone monitoring

In situ, real-time, continuous measurement of nutrients and metabolites, where the continuous measurement system is inserted/placed directly into the environment where the target analytes exist, e.g., in vivo monitoring or in situ fermentation monitoring systems, is ideal because it allows for more effective feedback control because of low or no time-lag, and eliminates concerns regarding contamination in sampling operations [Vojinovic (2006)]. Although the development of in situ, continuous, and real-time monitoring is expected, most of the currently available sensors are biophysical sensors, except the ones for glucose monitoring. Continuous glucose monitoring (CGM) systems are achieved with the combination of excellent biological or abiological molecular recognition elements and electrochemical or optical measurement principles [Lee (2021)].

BHBDhs have been commonly used for various detection systems, including spectroscopic analyses for central laboratory testing and handheld meter equipped with the electrochemical enzyme sensor strips. Recently, continuous BHB monitoring, or continuous ketone monitoring (CKM), is focused as the challenging area for the precise monitoring and care of diabetic patients, especially for those suffering from ketoacidosis. Biosensors for BHB have been reported from varieties of companies, and also reported in various academic researches. However, most of the sensors are for single use disposable electrochemical sensors, not continuous monitoring of BHB.

There are two major technological hurdles in the way of realizing CKM systems based on enzymatic analysis. Both are based on the limitation in the availabilities in the enzymes which can be used for the sensors.

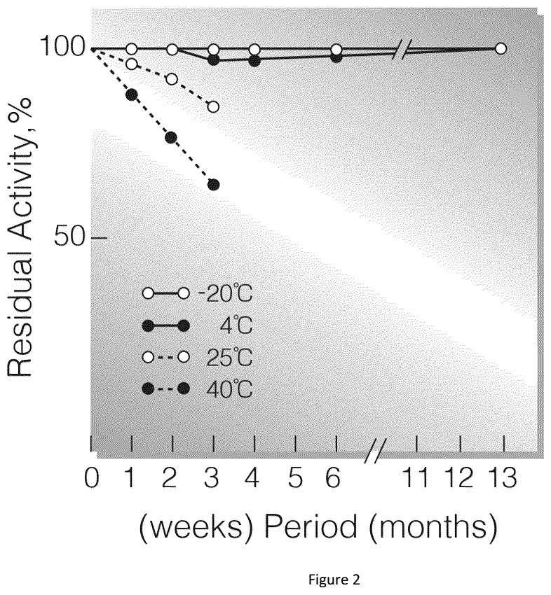

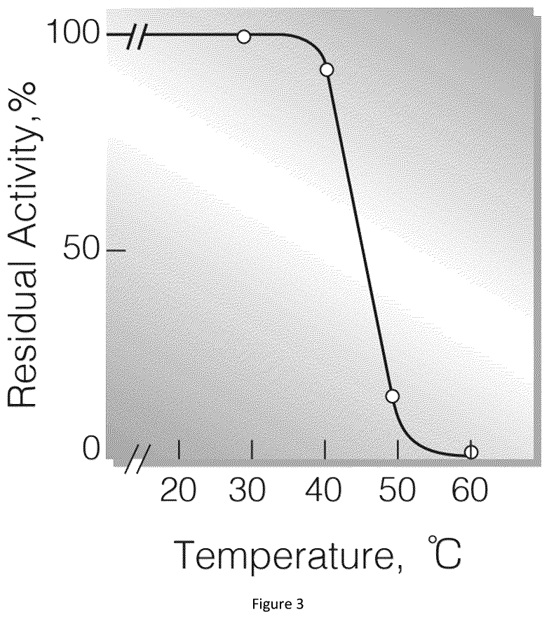

The first limitation is that the stability of the enzyme is not enough to be applied for continuous monitoring systems. There have been limited number of resources for BHBDhs, and those derived from Alcaligenes faecalis or Pseudomonas species, a mesophilic bacteria derived BHBDh have been used. These enzymes are stable in powder (dried) form for several weeks at 20° C. (FIG. 2); however, even in the powder form, its activity drastically decreased even at 25° C. Considering these stabilities under dried conditions, continuous operation in solution at around 37° C. for 1-2 weeks, which is necessary in order to be suitable for CKM, won't be possible with the enzyme derived from mesophiles.

The second limitation is the type of co-factor of BHBDh. Unlike the currently utilized enzyme for continuous glucose monitoring, glucose oxidase, which harbors its redox cofactor (flavin adenine dinucleotide; FAD) tightly bound in its enzyme molecule, BHBDh requires NAD⁺ for the oxidation of BH. NAD⁺ does not bind to the enzyme, and must be added in the reaction solution.

There have been only two reports about the construction of continuous ketone monitoring systems using BHBDh [Teymourian (2020); Alva (2021)]. One group reported a microneedle-based continuous ketone monitoring system [Teymourian (2020)]. They developed a functional multilayer reagent-coated microneedle biosensor, employing an ionic liquid (IL) based carbon paste transducer electrode incorporated with the phenanthroline-dione mediator, followed by a mixed BHBDh/NAD⁺ layer, glutaraldehyde cross-linking, and further coating with chitosan and polyvinyl chloride as outer polymer layers. The employment of IL, an imidazolium-based cation and trifluoromethyl sulfonylimide anionic groups, plays significant role in the stabilizing and confining NAD⁺ through hydrogen bonding along with coulombic interactions and imparting fouling-resistant and highly sensitive anodic detection of NADH. Another group [Alva (2021)] reported a single clinical site in vivo study with the continuous ketone sensor. According to their research article, they constructed ketone sensor with BHBDh, similar with their glucose sensor. There is no indication of how NAD is retained in the electrode. This reference teaches that a proprietary redox mediation chemistry containing osmium complex was deposited on the sensing layer to allow for selective oxidation of BHB and transport of electrons from the enzyme to the electrode using the wired enzyme technology. This sensor was reportedly stable for 14 days of continuous ketone monitoring in ISF with the healthy subjects. The in vitro study revealed that the sensor response covers a BHB range up to 8 mM, similar to their commercial product disposable ketone sensor.

Solving the above two technological limitations is necessary to realize continuous monitoring of BHB so as to be able to develop CKM systems.

In summary, the current biggest challenge is the limited availability of molecular recognition molecules for BHB monitoring; only NAD-dependent dehydrogenases which require soluble NAD⁺ in the reaction are now available, and are not suitable for CKM. To achieve CKM with extended lifetime and accuracy, new molecular recognition molecules for BHB, that can operate free of soluble cofactors, are necessary.

To overcome the above mentioned two technological limitations to realize in situ, real-time continuous ketone monitoring, novel BHBDhs have been developed: 1) thermostable BHBDhs, and 2) BHBDhs harboring tightly bound NAD in their molecule, the nicotinoprotein BHBDHs.

Thermostable BHBDhs; Based on the structural information of currently commercially available BHBDh, A. faecalis derived BHBDh, putative enzymes which are homologues of A. faecalis derived BHBDh were found, derived from various bacteria, including thermophilic bacteria and radiation-resistant bacteria, although none of them have been purified, cloned, recombinantly produced, or characterized before. The putative BHBDhs derived from Thermus thermophilus, Geobacillus stearothermophilus and Deinococcus radiotolerans were prepared, and it was found that they all possess BHBDh activity. Moreover, they are highly stable. As discussed in more detail below, whereas A. faecalis derived BHBDh starts becoming inactivated at temperatures higher than 40° C. and is totally inactivated by 20 min incubation at 55° C., BHBDhs derived from T. thermophilus, G. stearothermophilus and D. radiotolerans, were not inactivated at 40° C., and remained 100% (T. thermophilus and G. stearothermophilus) or more than 50% (D. radiotolerans) of initial activities even after incubation for 20 min at 55° C. Moreover, it was found that after incubation at 55-65° C. (T. thermophilus and G. stearothermophilus) or at 35-45° C. (D. radiotolerans), the enzyme activities increased 1.5-4 times, before the incubation. Moreover, introduction of mutations at Ala175Cys/Ala177Gly/Tyr178Trp of BHBDh derived from T. thermophilus resulted in a 3-fold increase in its catalytic activity. These results showed that novel and stable BHBDhs suitable for point of care ketone sensing and CKM systems had been developed.

Nicotinoprotein BHBDHs

A number of alcohol/aldehyde oxidoreductases have been discovered which have bound NAD(P) as a cofactor. These enzymes have been named as “Nicotino-enzymes” or “Nicotinoproteins”. They contain an intrinsic, tightly bound NAD(P)(H) that does not dissociate to exchange with the pool of free NAD(P)(H) in solution, however there are no report about nicotinoprotein BHBDHs. Engineered BHBDhs which harbor NAD in molecules were constructed by inserting a loop region derived from one of the nicotinoproteins, carveol dehydrogenase, and by introducing mutations into A. faecalis derived BHBDh, BHBDhs harboring NAD in their molecules were constructed. Such a NAD-bound engineered BHBDh is ideal for CKM systems.

The disclosed subject matter is further described in the following non-limiting Examples. It should be understood that these Examples, while indicating preferred embodiments of the invention, are given by way of illustration only.

### Example 1—Preparation of BHBDhs

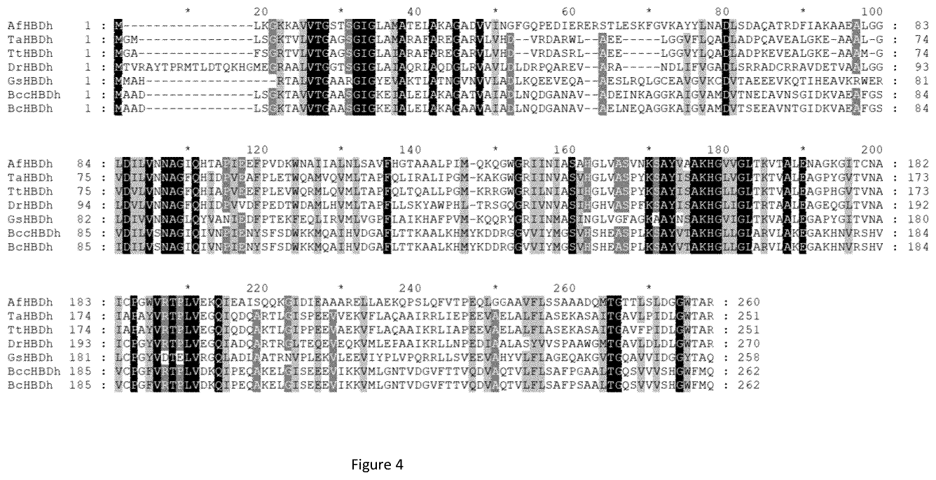

The putative amino acid sequences of BHBDhs derived from certain organisms are shown in FIG. 4. These hypothetical proteins had never been expressed and had their gene products characterized, and their functions were not known. Putative BHBDh sequences from the following organisms are shown:

AfBHBDh (or AfHBDh): Alcaligenes faecalis (WP_042488285.1)
(SEQ ID NO: 1)
MLKGKKAVVTGSTSGIGLAMATELAKAGADVVINGFGQPEDIERERST
LESKFGVKAYYLNADLSDAQATRDFIAKAAEALGGLDILVNNAGIQHTAPIEEFPV
DKWNAIIALNLSAVFHGTAAALPIMQKQGWGRIINIASAHGLVASVNKSAYVAAK
HGVVGLTKVTALENAGKGITCNAICPGWVRTPLVEKQIEAISQQKGIDIEAAAREL
LAEKQPSLQFVTPEQLGGAAVFLSSAAADQMTGTTLSLDGGWTAR
TaHBHDh (or TaHBDh): Thermus aquaticus (WP_003045371.1)
(SEQ ID NO: 2)
MGMLSGKTVLVTGAGSGIGLAMARAFAREGARVLVHDVRDARWLAE
ELGGVFLQADLADPQAVEALGKEAAALGVDILVNNAGFQHIDPVEAFPLETWQA
MVQVMLTAPFQLIRALIPGMKAKGWGRIINVASVHGLVASPYKSAYISAKHGLVG
LTKTVALEAGPYGVTVNAIAPAYVRTPLVEGQIQDQARTLGISPEEVVEKVFLAQ
AAIRRLIEPEEVAELALFLASEKASAITGAVLPIDLGWTAR
TtBHBDh (or TtHBDh): Thermus thermophiles (WP_014510095.1)
(SEQ ID NO: 3)
MGAFSGRTVLVTGAGSGIGLAIARAFAREGARVLVHDVRDASRLAEEL
GGVYLQADLADPKEVEALGKEAAAMGVDVLVNNAGFQHIAPVEEFPLEVWQRM
LQVMLTAPFQLTQALLPGMKRRGWGRILNIASIHGLVASPYKSAYISAKHGLLGL
TKTVALEAGPHGVTVNAIAPAYVRTPLVEGQIQDQAKTLGIPPEEVAEKVFLAQA
AIKRLIAPEEVAELALFLASEKASAITGAVFPIDLGWTAR
DrBHBDh (or DrHBDh): Deinococcus radiotolerans (GGK93151.1)
(SEQ ID NO: 4)
MTVRAYTPRMTLDTQKHGMEGRAALVTGGTSGIGLAIAQRLAQDGLR
VAVLDLDRPQAREVARANDLIFVGADLSRRADCRRAVDETVAALGGLDVLVNN
AGFQHIDPVVDFPEDTWDAMLHVMLTAPFLLSKYAWPHLTRSGQGRIVNVASIH
GHVASPFKSAYISAKHGLIGLTRTAALEAGEQGLTVNAICPGYVRTPLVEGQIADQ
ARTRGLTEQEVEQKVMLEPAAIKRLLNPEDIAALASYVVSPAAWGMTGAVLDLD
LGWTAR
GsBHBDh (or GsHBDh): Geobacillus stearothermophilus (KZE97055.1)
(SEQ ID NO: 5)
MMAHRTALVTGAARGIGYEVAKTLATNGVNVVLADLKQEEVEQAAE
SLRQLGCEAVGVKCDVTAEEEVKQTIHEAVKRWERLDIVVNNAGLQYVANIEDF
PTEKFEQLIRVMLVGPFLAIKHAFPVMKQQRYGRIINMASINGLVGFAGKAAYNS
AKHGVIGLTKVAALEGAPYGITVNALCPGYVDTELVRGQLADLAATRNVPLEKV
LEEVIYPLVPQRRLLSVEEVAHYVLFLAGEQAKGVTGQAVVIDGGYTAQ
BccBHBDh (or BccHBDh): Burkholderia cenocepacia (WP027810191.1)
(SEQ ID NO: 6)
MAADLSGKTAVVTGAASGIGKEIALELAKAGATVAIADLNQDGANAV
ADEINKAGGKAIGVAMDVTNEDAVNSGIDKVAEAFGSVDILVSNAGIQIVNPIENY
SFSDWKKMQAIHVDGAFLTTKAALKHMYKDDRGGVVIYMGSVHSHEASPLKSA
YVTAKHGLLGLARVLAKEGAKHNVRSHVVCPGFVRTPLVDKQIPEQAKELGISEE
EVIKKVMLGNTVDGVFTTVQDVAQTVLFLSAFPGAALTGQSVVVSHGWFMQ
BcBHBDh (or BcHBDh): Burkholderia cepacian (WP_034207826.1)
(SEQ ID NO: 7)
MAADLSGKTAVVTGAASGIGKEIALELAKAGAAVAIADLNQDGANAV
AELNEQAGGKAIGVAMDVTSEEAVNTGIDKVAEAFGSIDILVSNAGIQIVNPIENY
SFSDWKKMQAIHVDGAFLTTKAALKHMYKDDRGGVVIYMGSVHSHEASPLKSA
YVTAKHGLLGLARVLAKEGAKHNVRSHVVCPGFVRTPLVDKQIPEQAKELGISEE
EVIKKVMLGNTVDGVFTTVQDVAQTVLFLSAFPSAALTGQSVVVSHGWFMQ

FIGS. 28A and 28B depict the alignments of the above sequences, along with additional putative sequences of BHBDh homologs from the following organisms:

Achromobacter (WP_049071820.1)
(SEQ ID NO: 12)
MLKGKVAVVTGSTSGIGLGIATALAAQGADIVLNGFGDAAEIEKVRAG
LAAQHGVKVLYDGADLSKGEAVRGLVDNAVRQMGRIDILVNNAGIQHTA
LIEDFPTEKWDAILALNLSAVFHGTAAALPHMKKQGFGRIINIASAHGL
VASANKSAYVAAKHGVVGFTKVTALETAGQGITANAICPGWVRTPLVEK
QISALAEKNGVDQETAARELLSEKQPSLQFVTPEQLGGTAVFLASDAAA
QITGTTVSVDGGWTAR
Cereibacter spaeroides KD131 (ACM00960.1)
(SEQ ID NO: 13)
MHMTGGRIMDLNGKRAIVTGSNSGIGLGCAEELARAGAEVVINSFTDR
DEDHALAEKIGREHGVSCRYIAADMSDGEACRALIETAGGCDILVNNAG
IQHVSSIEEFPVEKWNAILAINLSSAFHTTAAALPGMRAKGWGRIVNIA
SAHGLTASPYKSAYVAAKHGVVGFTKVTALETAGKGITCNAICPGYVLT
PLVEAQIPDQMKAHDMDRETVIREVMLDRQPSRQFATTGQIGGTVVFLC
SGAADQITGTTISVDGGWTAL
Paucimonas lemoignei (WP_132256256.1)
(SEQ ID NO: 14)
MQGKTLQGKTALVTGSTSGIGLGIARSLAEAGANIVENGFGDQKEIEAL
QQSVAKEFGVQTAYHNADMSKASEIEALMKFAAEKFGMVDVLVNNAGIQ
HVANVEDFPVEKWDAIIAINLTSAFHTTRLALPAMKAKNWGRIINIASV
HGLVGSAQKSAYVAAKHGIVGLTKVSALENAQTGVTVNAICPGWVLTPL
VQKQVDARAAANNQTNDEAKRQLLLEKQPSGEFVTPEQLGSLAVYLCSD
AASQMRGMSLNVDGGWVAQ
Burkholderiaceae (WP_062171810.1)
(SEQ ID NO: 15)
MTSSAATPLAGKTALVTGSTSGIGLGIANALAQAGANIVLNGFGDATVI
QNAKSQIEQHGVKAVHHGADMSKATEIEAMIAFAIEQFGAVDVLVNNAG
IQHVATIDTFPVEKWDAIIAINLTSAFHTMRVALPKMRDNGWGRVINIA
SAHGLVGSVGKSAYVAAKHGIVGLTKVAALENARTGVTVNAICPGFVLT
PLVQKQIDDIAAKENISPDAARSKLLGEKQPSEQFVTPEQIGKLAVFLC
SEAADEMRGSALQIDGGWTSQ
Citrobacter (WP_003843689.1)
(SEQ ID NO: 16)
MNLTGKTALVTGSTSGIGLGIAQVLAQAGATLILNGFGDVDAAKDAV
AQYGKTPGYHGADLSDEAQIADMMRYAESEFGGVDILINNAGIQHVSPI
ETFPVDKWNAIIAINLSSVFHTTRLALPGMRARNWGRIINIASVHGLVA
SKEKSAYVAAKHGVVGLTKTIALETAQTEITCNALCPGWVLTPLVQQQI
DKRIAEGAEPEAARDALLAEKQPSREFVTPEQLGNLALFLCSDGAAQVR
GVAWNMDGGWVAQ
Acinetobacter calcoaceticus (WP_000163845.1)
(SEQ ID NO: 17)
MTKLLDGKVAFITGSASGIGLEIAKKFAQEGAKVVISDMNAEKCQETA
NSLKEQGFDALSAPCDVTDEDAYKQAIELTQKTFGTVDILINNAGFQHV
APIEEFPTAVFQKLVQVMLTGAFIGIKHVLPIMKAQKYGRIINMASING
LIGFAGKAGYNSAKHGVIGLTKVAALECARDGITVNALCPGYVDTPLVR
GQIADLAKTRNVSLDSALEDVILAMVPQKRLLSVEEIADYAIFLASSKA
GGVTGQAVVMDGGYTAQ
Psychrobacter immobilis (WP_201584272.1)
(SEQ ID NO: 18)
MNTYFSELKNKTVVITGSSKGIGKNIALTFAKLEANVVISGRDEATLDA
VLKEMHKYNSKCIAVSGNLSDITQIRNLIDKAASQFGTIDVLVNNAGVN
IAKPAMEVTEEDWDAVLDLNLKTAFFASQSAAKYMLKQNNGRIINIASQ
MAFVGYVKRAAYCSSKGGLVQLTKALAVEWAKQGIRVNAVAPTFIETEL
TEKMFIDEAFKKDVDSRILLDGLSQPEDISGAVLYLASNLANFVTGETI
KVDGGWTAI
Psychrobacter arenosus (WP_201497461.1)
(SEQ ID NO: 19)
MATQLTQDLTGQVALVTGSASGIGRDIAETYAKAGATVGIADINIEAA
QKVVDAIESQGGKALAIEMDVTSEDAVNAGVQKLVDSFGSIDILVSNAG
IQIIDPINKMSYDNWKKMLAIHLDGAFLTTKAALQHMYQNDKVGTVIYM
GSVHSHEASMYKAPYVTAKHGLLGLCRVLAKEGAEHNVRAHVICPGFVK
TPLVEKQIPEQAAEKGISEEAVVNDIMLVNTVDKEFTTVDDIAQLALFL
AAFPSNVFTGQSIVASHGWFMN
Psychrobacter arcticus (WP_011280696.1)
(SEQ ID NO: 20)
MATQLQQDLTGKVALVTGAASGIGRDIAETYAKAGAAVGIADINLEA
AQKTVDAIEAAGGRALAIAMDVTSEAAVNDGVQRLVDTFGGIDILVSNA
GIQIIDPIHKMAFEDWKKMLAIHLDGAFLTTKAAIQHMYKDDKGGTVIY
MGSVHSHEASLFKAPYVTAKHGLLGLCRVLAKEGAVHNVRSHVICPGFV
KTPLVEKQIPQQAAEKGISEESVVNDIMLVNTVDKEFTTVDDIAQLALF
LAAFPTNVFTGQSIVASHGWFMN

The sequence alignments demonstrate how the positions of corresponding amino acid residues may be numbered differently in BHBDh sequences derived from different organisms. For example, position 175 in SEQ ID NO: 3 (TtBHBDh) corresponds to position 184 in SEQ ID NO: 1 (AfBHBDh), and to position 194 in SEQ ID NO: 4 (DrBHBDh).

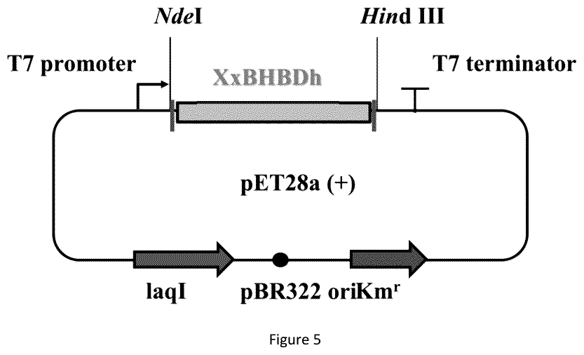

FIG. 5 shows the vector plasmids for the expression of putative BHBDhs derived from Thermus thermophilus, Geobacillus stearothermophilus and Deinococcus radiotolerans.

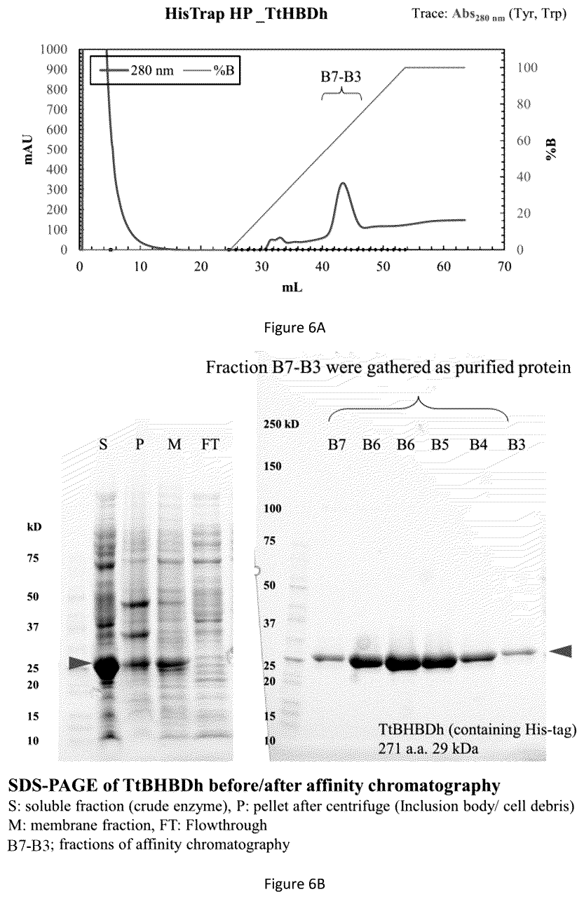

FIG. 6A shows the purification of putative BHBDh derived from Thermus thermophilus (TtBHBDh) (sample: crude enzyme ˜3 mL=50 mL culture). The purification was performed on a His Trap HP (1 mL) column. The A buffer was 10 mM Imidazole, 500 mM NaCl, and 10 mM HEPES pH 8.0. The B buffer was 500 mM Imidazole, 500 mM NaCl, and 10 mM HEPES pH 8.0. The gradient used was B 0-100%/30 CV. Finally, the sample was BHBDh from Thermus thermophilus (crude enzyme ˜3 mL=50 mL culture).

FIG. 6B shows SDS PAGE analyses of the putative TtBHBDH, before (left) and after (right) purification.

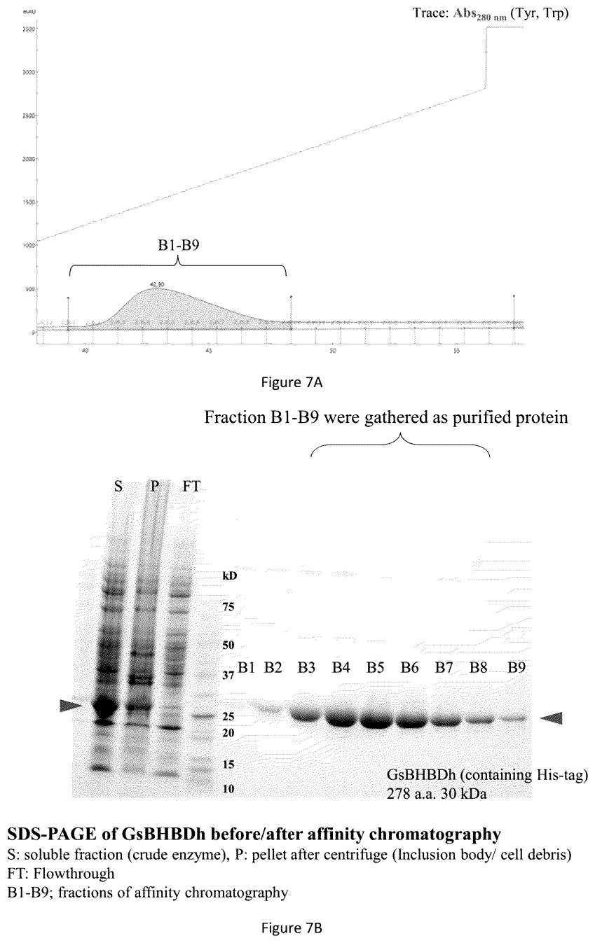

FIG. 7A shows the purification of putative BHBDh derived from Geobacillus stearothermophilus (GsBHBDh) (sample: crude enzyme ˜5.6 mL=100 mL culture). The A buffer, B buffer, gradient, and column were the same as used with the TtBHBDH.

FIG. 7B shows SDS PAGE analyses of the putative GsBHBDH, before (left) and after (right) purification.

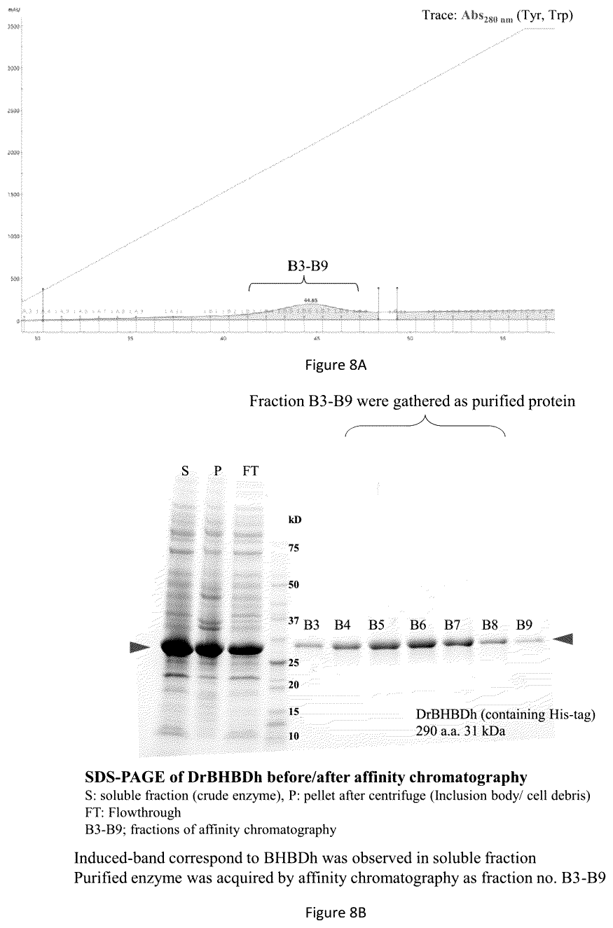

FIG. 8A shows the purification of putative BHBDh derived from Deinococcus radiotolerans (DrBHBDh) (sample: crude enzyme ˜5.6 mL=100 mL culture). The A buffer, B buffer, gradient, and column were the same as used with the TtBHBDH.

FIG. 8B shows SDS PAGE analyses of the putative DrBHBDH, before (left) and after (right) purification.

In sum, the results in FIGS. 6A-8B demonstrate the recombinantly produced putative proteins, derived from Thermus thermophilus, Geobacillus stearothermophilus and Deinococcus radiotolerans have been successively purified with expected molecular weights.

Table 1, below, shows the BHB dehydrogenase activities of recombinantly prepared putative BHBDHs FIGS. 6A-8B, determined by monitoring increase of NADH concentration using NAD as the cofactor, at room temperature. These results showed that the putative BHBDhs derived from Thermus thermophilus, Geobacillus stearothermophilus and Deinococcus radiotolerans are all BHBDhs, and are compared to the activity of A. faecalis. Activities were measured using absorbance at 340 nm, in a solution of 1 mM NAD⁺, 50 mM HB, 10 mM HEPES at pH 7.8, and 10 mM MgSO₄.

TABLE 1
BHB Dehydrogenase activities of putative BHBDHs derived from
Thermus thermophilus, Geobacillus stearothermophilus and
Deinococcus radiotolerans compared to A. faecalis

| Sample | Concentration (mg/mL) | Activity (U/mg) | Productivity (U/L) |
|---|---|---|---|
| GsBHBDh | 2.01 | 11.84 | 1324 |
| DrBHBDh | 0.096 | 21.58 | 480 |
| TtBHBDh | 1.40 | 3.711 | 89 (purified) |
| AfBHBDh | 0.904 | 256.8 | 26247 (purified) |

### Example 2—BHBDh Activity

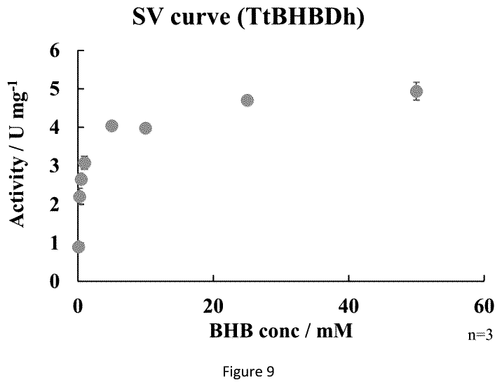

FIG. 9 shows BHB concentration-dependent BHBDh activity of recombinantly prepared BHBDhs derived from Thermus thermophilus (TtBHBDh). The activity was measured using NADH detection via absorbance at 340 nm, at room temperature. The measurement was done in a solution with 1 mM NAD⁺, 10 mM HEPES at pH 7.8, and 0 mM MgCl₂·6 H₂O, from 0-50 mM BHB. This enzyme shows a very low Kₘ value (0.359 mM) toward BHB, which indicates this enzyme is suitable for BHB monitoring. The activity also has a Vₘₐₓ of 4.54 U/mg, and a Vₘₐₓ/Kₘ of 12.6.

### Example 3—Thermal Stability

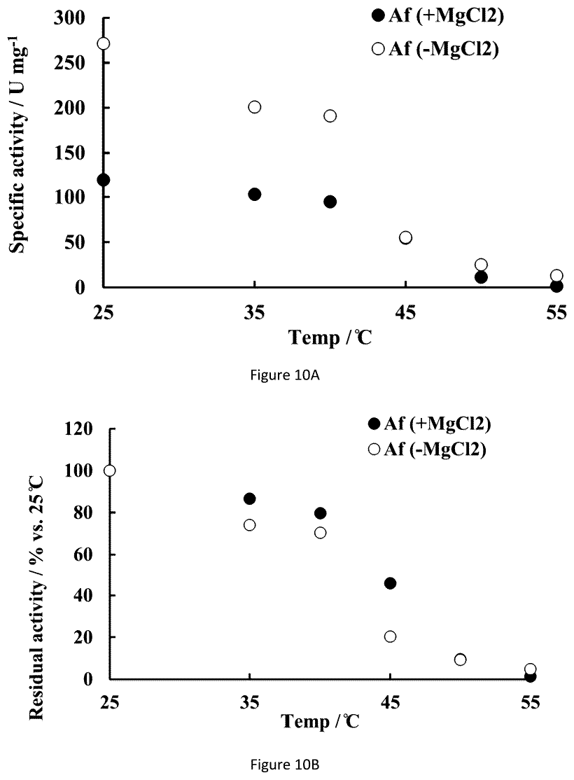

Next, the thermal stability of some of the new BHBDhs were tested, and compared to the commercially available BHBDh. FIGS. 10A and 10B show the thermal stability of BHBDh derived from Alcaligenes faecalis (AfBHBDh), using specific activity and relative residual activity, respectively. The measurements were performed by 20 minutes of heat treatment at each temperature, using NADH detection (absorbance at 340 nm). in a solution with 1 mM NAD, 50 mM BHB, 10 mM HEPES at pH 7.8, and either 0 or 10 mM MgCl₂·6 H₂O.

Similarly to the commercially available one shown in FIG. 2, AfBHBDh decreased its activity by incubation at higher temperature than 40° C., and was completely inactivated at 55° C. These results supported the premise that commercially available BHBDhs are thermally labile and unstable.

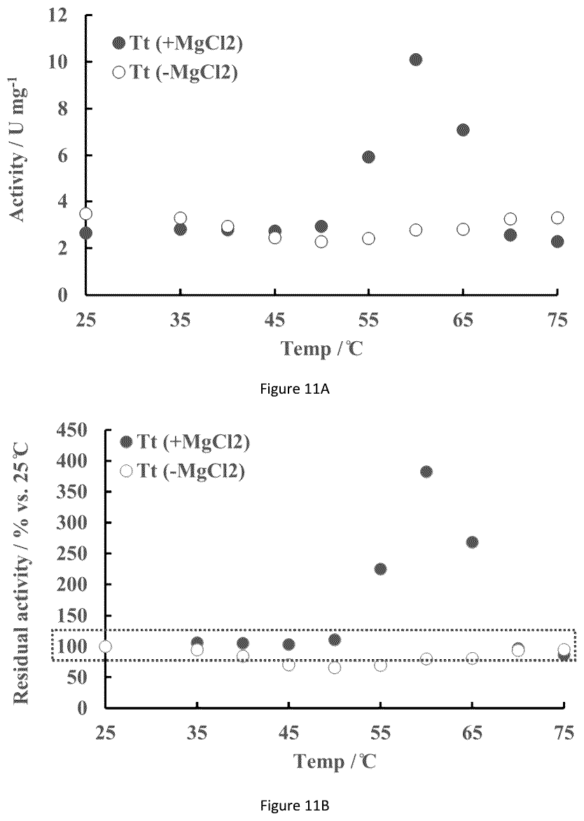

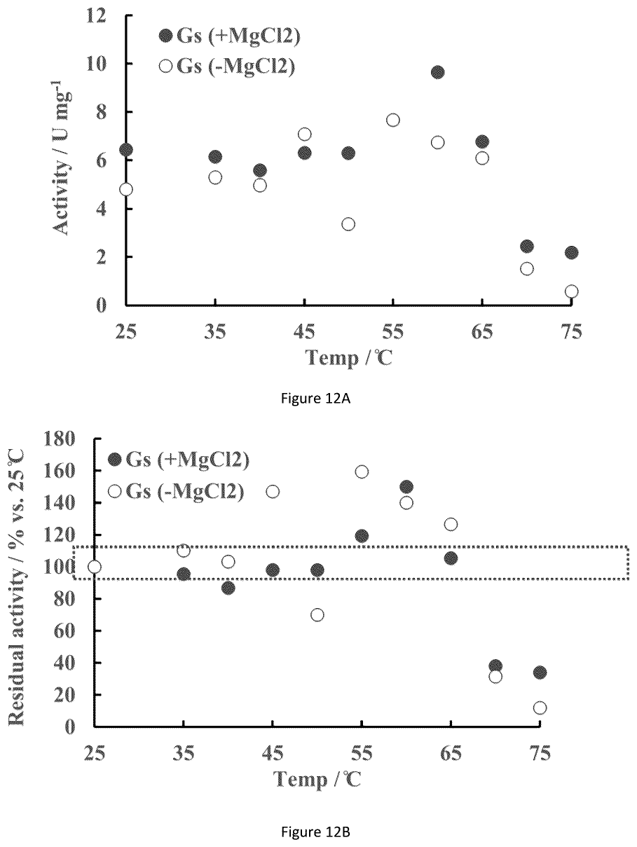

Under identical reaction conditions, the same testing was performed on TtBHBDh, and results are shown in FIGS. 11A and 11B. Unlike the commercially available BHBDhs shown in FIGS. 2 and 10, TtBHBDh was not inactivated at heat treatments investigated (25-75° C.), and even after a 20 min incubation at 75° C., TtBHBDh retained 100% of its enzyme activity. Moreover, the heat treatment at temperatures between 55-65° C. in the presence of 10 mM MgCl₂, resulted in increased enzyme activity (by up to 400%). These results show that TtBHBDh harbors hyper thermal stability, and is suitable for BHB POCT devices and CKM monitoring.

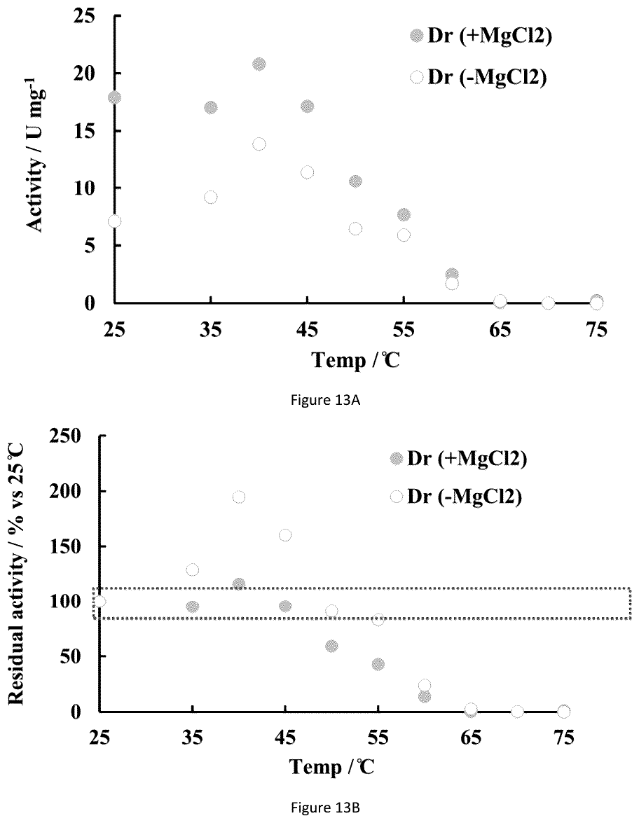

Under identical reaction conditions, the same testing was also performed on GsBHBDh, and results are shown in FIGS. 12A and 12B. Unlike the commercially available BHBDhs shown in FIGS. 2 and 10, GsBHBDh was not inactivated at heat treatment investigated (25-65° C.), and even after a 20 min incubation at 65° C., GsBHBDh retained 100% of its enzyme activity. Moreover, the heat treatment at temperatures between 55-60° C. in the presence of 10 mM MgCl₂ resulted in increased enzyme activity (up to 140%). These results show that GsBHBDh harbors hyper thermal stability, and is suitable for BHB POCT devices and CKM monitoring.

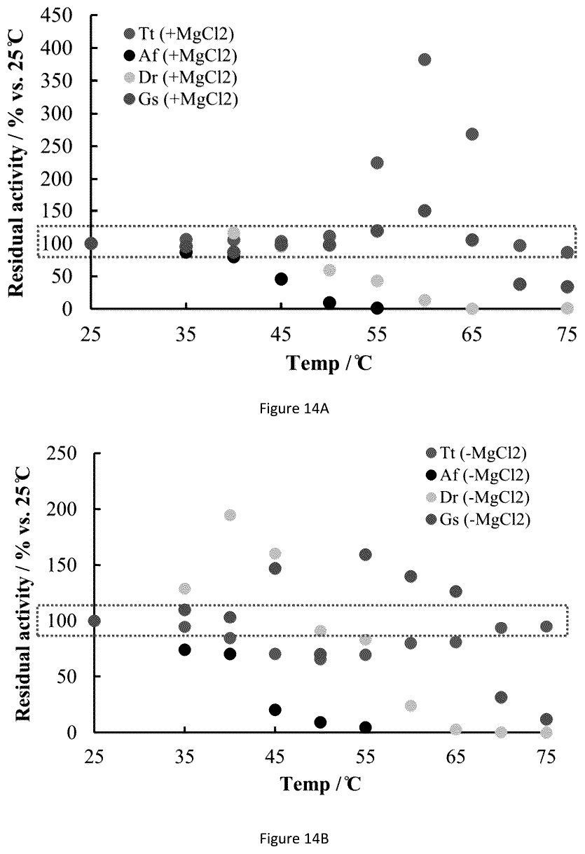

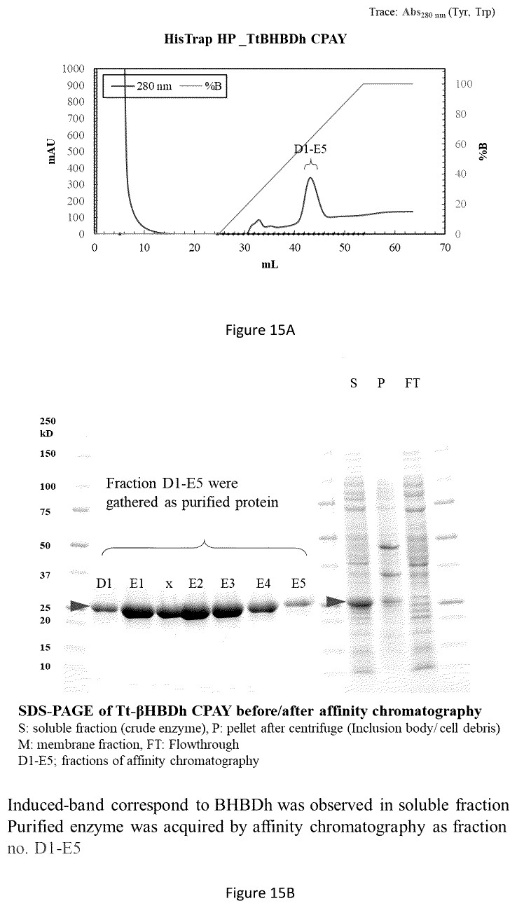

Finally, under identical reaction conditions, the same testing was also performed on DrBHBDh, and results are shown in FIGS. 13A and 13B. Unlike the commercially available BHBDhs shown in FIGS. 2 and 10, DrBHBDh was not inactivated at heat treatment investigated (25-45° C.), and even after the 20 min incubation at 45° C., DrBHBDh retained 100% of its enzyme activity. Moreover, the heat treatment at temperature between 35-45° C. in the presence or absence of 10 mM MgCl₂, resulted in increased enzyme activity (up to 200%). These results show that DrBHBDh harbors thermal stability, and is suitable enzyme for BHB POCT devices and CKM monitoring.

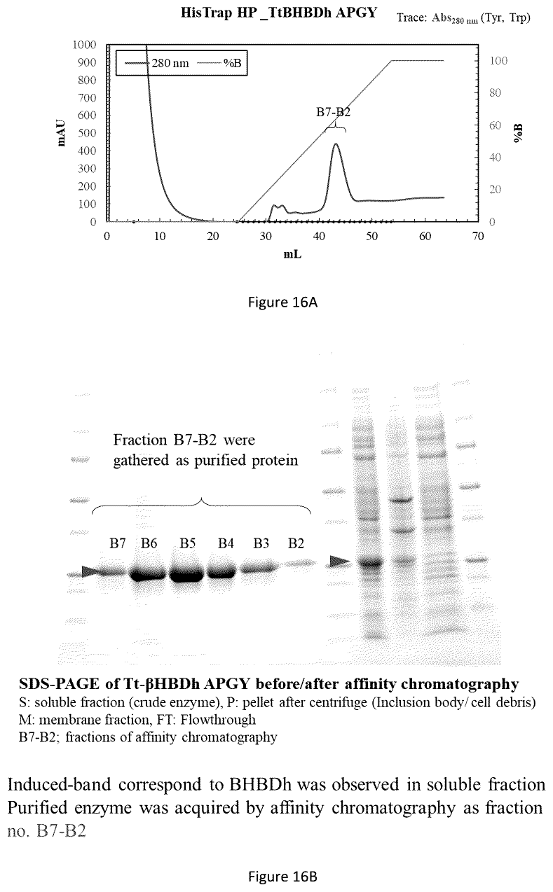

FIG. 14A shows compiled specific activity data for AfBHBDh, TtBHBDh, GsBHBDh, and DrBHBDr, in the presence of 10 mM MgCl₂. FIG. 14B shows compiled relative residual activity data for AfBHBDh, TtBHBDh, GsBHBDh, and DrBHBDr, in the absence of MgCl₂. Unlike the commercially available BHBDhs shown in FIGS. 2 and 10, newly discovered BHBDhs were not inactivated at 45° C., at which temperature the commercially available enzymes are inactivated. These results show that newly discovered BHBDhs derived from Thermus thermophilus (TtBHBDh), Geobacillys stearothermophilus (GsBHBDh) and from Deinococcus radiotolerans (DrBHBDh) harbor thermal stability, and are suitable for BHB POCT devices and CKM monitoring.

### Example 4—TtBHBDh Mutants

Preparation and Purification

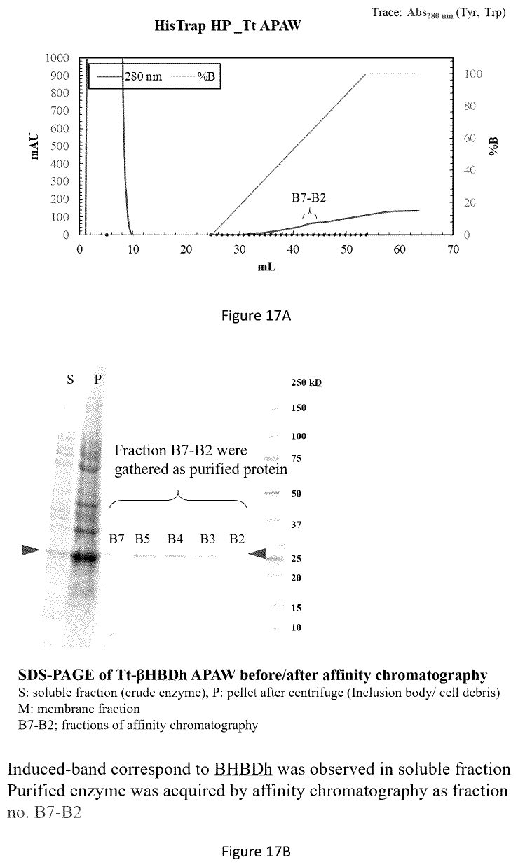

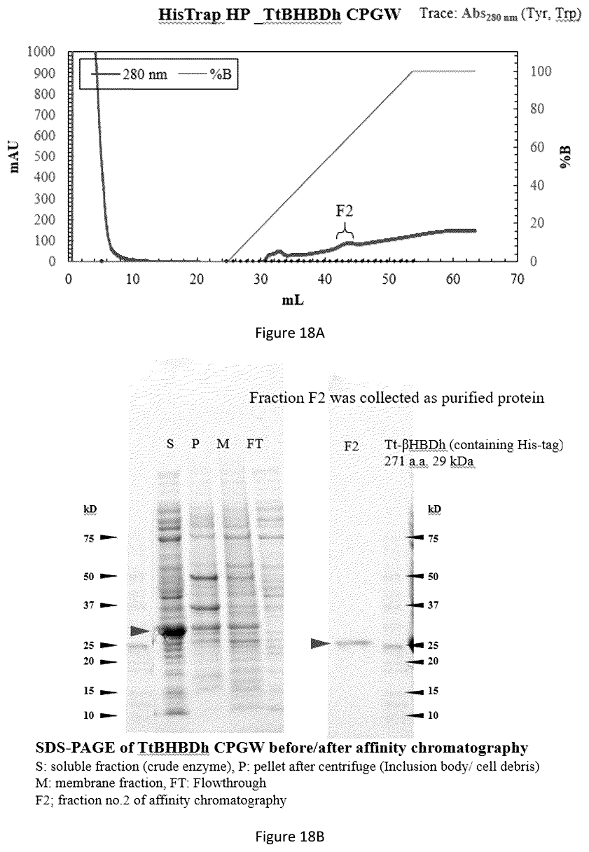

A number of different TtBHBDh mutants were prepared, and purified by affinity chromatography and SDS-PAGE. The mutants were Ala175Cys (results shown in FIGS. 15A-15B), Ala177Gly (FIGS. 16A-16B), Tyr178Trp (FIGS. 17A-17B), and Ala175Cys/Pro176/Ala177Gly/Tyr178Trp (CPGW) (FIGS. 18A-18B).

FIG. 15A shows the purification of the Ala175Cys TtBHBDh mutant via affinity chromatography (sample: crude enzyme ˜3 mL=100 mL culture). The column used was a His Trap HP (1 mL) column. The A buffer contained 10 mM imidazole, 500 nM NaCl, and 10 mM HEPES at pH 8.0, while the B buffer contained 500 mM imidazole, 500 nM NaCl, and 10 mM HEPES at pH 8.0. The gradient used was B 0-100%/30 CV. The peak was observed around 40-60% B. FIG. 15B shows results of SDS-PAGE analysis of the Ala175Cys sample (CPAY) before (left) and after (right) affinity chromatography.

FIG. 16A shows the purification of the Ala177Gly TtBHBDh mutant via affinity chromatography, using the same column, buffers, and gradient as in FIG. 15A. The peak was observed around 40-60% B. FIG. 16B shows results of SDS-PAGE analysis of the Ala177Gly sample (APGY) before (left) and after (right) affinity chromatography.

FIG. 17A shows the purification of the Tyr178Trp TtBHBDh mutant via affinity chromatography, using the same column, buffers, and gradient as in FIG. 15A. A weak peak was observed around 50% B. FIG. 17B shows results of SDS-PAGE analysis of the Tyr178Trp sample (APAW) before (left) and after (right) affinity chromatography.

FIG. 18A shows the purification of the CPGW TtBHBDh mutant via affinity chromatography, using the same column, buffers, and gradient as in FIG. 15A. A weak peak was observed around 50% B. FIG. 18B shows results of SDS-PAGE analysis of the CPGW sample before (left) and after (right) affinity chromatography.

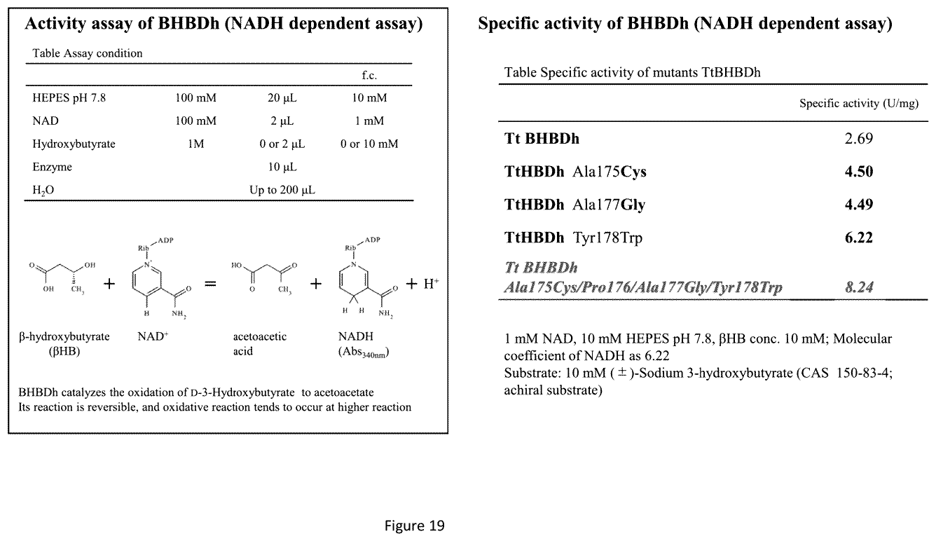

FIG. 19 shows the enzyme activities of wild type and mutant TtBHBDhs; Ala175Cys, Ala177Gly, Tyr178Trp and Ala175Cys/Pro176/Ala177Gly/Tyr178Trp. Compared with the wild type enzyme (2.69 U/mg) all mutant enzymes showed increased activities; Ala175Cys; 4.50 U/mg (>150%), Ala177Gly; 4.49 U/mg (>150%), Tyr178Trp; 6.22 U/mg (>200%) and Ala175Cys/Pro176/Ala177Gly/Tyr178Trp; 8.24 U/mg (>300%). These results indicated that the introduction of mutations on Ala175, Ala177, or Tyr178, and their combination improve the enzyme activities.

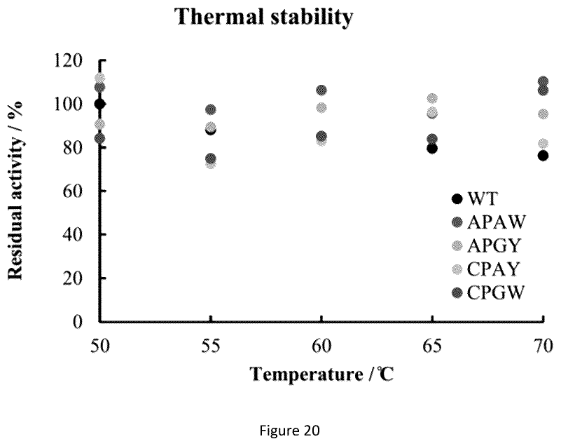

FIG. 20 shows the thermal stabilities of wild type and mutant TtBHBDhs; Ala175Cys, Ala177Gly, Tyr178Trp and Ala175Cys/Pro176/Ala177Gly/Tyr178Trp. For the thermal stability tests, 0.1 mg of protein/mL was prepared. The heat treatment used 50 μL of purified enzyme in a 0.2 mL PCR tube. The protein was held at each temperature for 20 minutes (no preheat). Each protein was then held on ice for more than two minutes. The activity measurement was tested for activity on NAD, using a solution of 1 mM NAD, 50 mM HB in 10 MM HEPES at pH 7.8. The measurements were obtained at 25° C. (plate reader).

The constructed mutant TtBHBDHs demonstrated thermal stabilities nearly identical to that of the wild type TtBHBDH, and they are not inactivated with the incubation at 75° C. for 20 min, indicated that these mutations resulted in the increase in enzyme activity by keeping their identical characteristic that TtBHBDh is thermally stable.

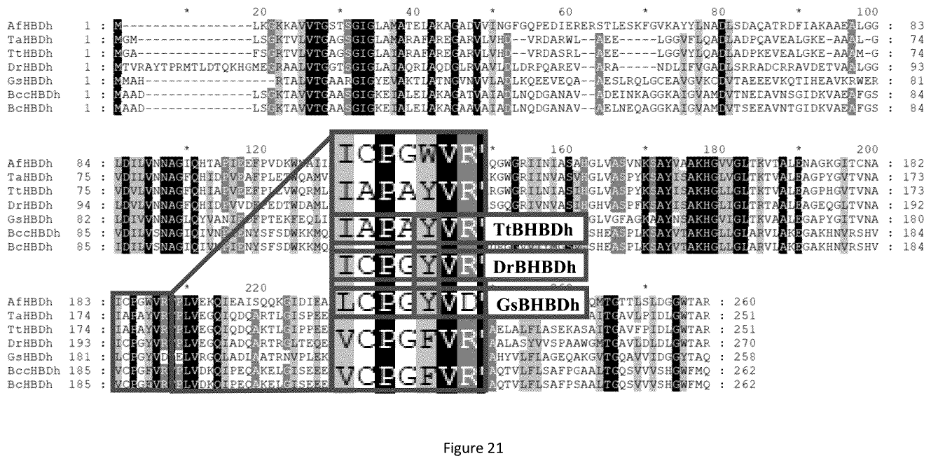

It was found that the mutation on Tyr residue, conserved in Thermus thermophilus (TtBHBDh), Geobacillys stearothermophilus (GsBHBDh) and from Deinococcus radiotolerans (DrBHBDh), improved the enzyme activities. FIG. 21 shows the amino acid sequence alignments of BHBDh derived from Alcaligenes faecalis (AfBHBDh), Thermus thermophilus (TtBHBDh), Geobacillys stearothermophilus (GsBHBDh) and from Deinococcus radiotolerans (DrBHBDh). As was shown in FIGS. 19 and 20, the Tyr178Trp mutation on TtBHBDh drastically increases enzyme activity. As are observed in FIG. 21, and in enlarged insert, Tyr178 is conserved in BHBDh of Geobacillys stearothermophilus (GsBHBDh) and BHBDh of Deinococcus radiotolerans (DrBHBDh). This indicates that amino acid substitution on conserved Tyr178 in GsBHBDh and in DrBHBDh will improve enzyme activities of these thermostable BHBDhs.

### Example 5—Loop Insertion

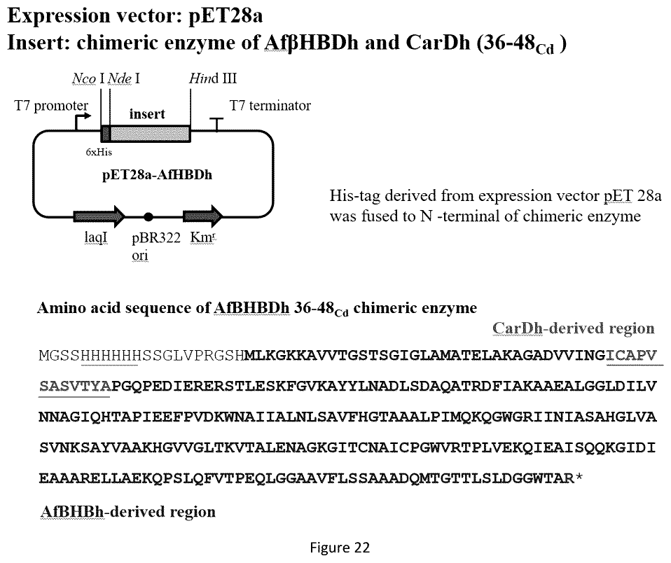

FIG. 22 shows the expression vector (above) inserted by a chimeric enzyme composed of AfBHBDh inserted by a loop region (12 amino acid residues, ICAPVSASVTYA (SEQ ID NO:10)) sequence derived from a nicotinoprotein dehydrogenase, carveol dehydrogenase derived from Mycobacterium tuberculosis, designated as AfBHBDh 36-48_{Cd} chimeric enzyme. Its amino acid sequence is as follows:

(SEQ ID NO: 8)
MLKGKKAVVTGSTSGIGLAMATELAKAGADVVINGICAPVSASVTYAP
GQPEDIERERSTLESKFGVKAYYLNADLSDAQATRDFIAKAAEALGGLD
ILVNNAGIQHTAPIEEFPVDKWNAIIALNLSAVFHGTAAALPIMQKQGW
GRIINIASAHGLVASVNKSAYVAAKHGVVGLTKVTALENAGKGITCNAI
CPGWVRTPLVEKQIEAISQQKGIDIEAAARELLAEKQPSLQFVTPEQLG
GAAVFLSSAAADQMTGTTLSLDGGWTAR

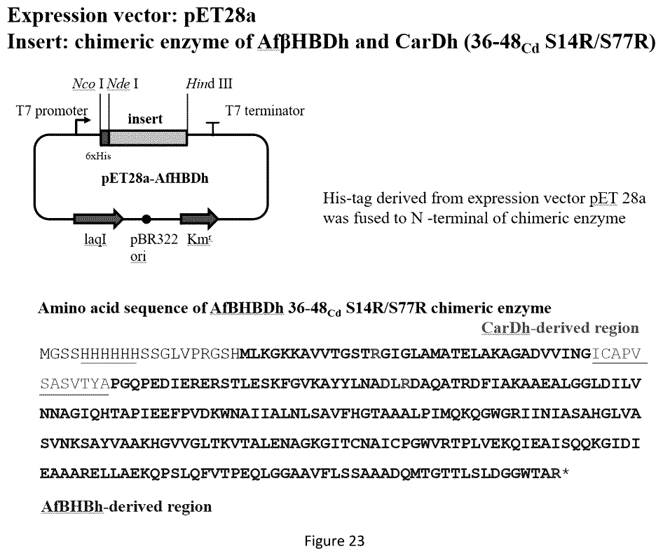

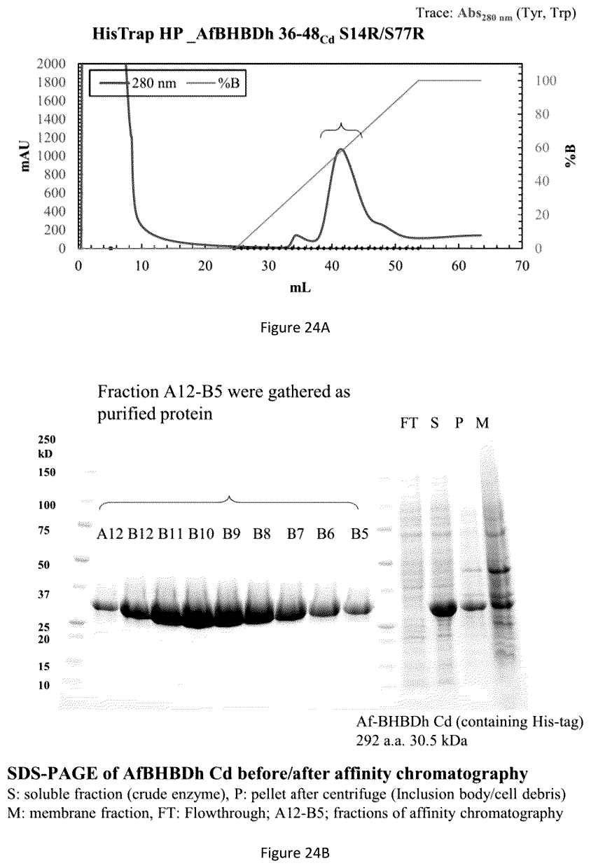

FIG. 23 shows the expression vector (above) inserted by a chimeric enzyme composed of AfBHBDh inserted by a loop region (12 amino acid residues, ICAPVSASVTYA (SEQ ID NO:10)) sequence with two amino acid substitutions; Ser14Arg/Ser71Arg, derived from a nicotinoprotein dehydrogenase, carveol dehydrogenase derived from Mycobacterium tuberculosis, designated as AfBHBDh 36-48_{Cd} S14R/S77R chimeric enzyme. Its amino amino acid sequence (with the S14R and S77R substitutions in bold) is as follows:

(SEQ ID NO: 9)
MLKGKKAVVTGSTRGIGLAMATELAKAGADVVINGICAPVSASVTYA
PGQPEDIERERSTLESKFGVKAYYLNADLRDAQATRDFIAKAAEALGGL
DILVNNAGIQHTAPIEEFPVDKWNAIIALNLSAVFHGTAAALPIMQKQG
WGRIINIASAHGLVASVNKSAYVAAKHGVVGLTKVTALENAGKGITCNA
ICPGWVRTPLVEKQIEAISQQKGIDIEAAARELLAEKQPSLQFVTPEQL
GGAAVFLSSAAADQMTGTTLSLDGGWTAR

In each of FIGS. 22 and 23, the AfBHBDh 36-48_{Cd} or AfBHBDh 36-48_{Cd} S14R/S77R chimeric enzyme has a His (6×)-tag on the N-terminal end (MGSSHHHHHHSSGLVPRGSH (SEQ ID NO:11)).

Cell Culture and Crude Enzyme Preparation

Expression vectors of pET28a inserted with either the AfBHBDh 36-48_{Cd} or AfBHBDh 36-48_{Cd} S14R/S77R chimeric enzyme harboring a His (6×)-tag on the N-terminal end, were transformed with E. coli LOBSTR-BL21(DE3). The transformed cells single colonies were pre-cultured into 3 mL LB with 50 μg/mL kanamycin under aerobic conditions (280 rpm) at 37° C. Overnight precultures were adjusted as OD660=3.0 and then inoculated as 1% v/v to ZYP-5052 medium (Autoinduction medium; Peptone140 (Fisher Scientific)) with 0.5% glycerol, 0.05% glucose, 0.2% lactose, 50 mM (NH₄)PO₄, 50 mM Na₂HPO₄, and 1 mM MgSO₄) containing 50 μg/mL kanamycin, and were cultivated at 25° C. for 24 h with rotation rate of 170 rpm. After the cultivation, cells were collected and washed with saline (0.85% NaCl).

Purification

The pellet was combined with an affinity chromatography buffer (3 mL/g wet cell) and then sonicated (Amp30, 15 seconds on followed by 45 seconds off, performed 3 times, for six rounds). The sonicated mixture was then centrifuged at 16,000 g at 4° C. for 20 minutes. The supernatant was separated from the pellet (containing the inclusion body and cell debris). The supernatant was centrifuged again at 16,000 g at 4° C. for 20 minutes. The supernatant (containing crude enzyme) was separated from the pellet (containing the membrane fraction). The supernatant was filtered and affinity chromatography was performed on a His Trap HP (1 mL) column. The A buffer contained 10 mM imidazole, 500 nM NaCl, and 10 mM HEPES at pH 8.0, while the B buffer contained 500 mM imidazole, 500 nM NaCl, and 10 mM HEPES at pH 8.0. The gradient used was B 0-100%/30 CV.

FIGS. 24A and 24B show the purification of AfBHBDh 36-48_{Cd} S14R/S77R chimeric enzyme (FIG. 24A) (crude enzyme ˜5 mL), and SDSPAGE analyses, before and after affinity chromatography) (FIG. 24B), as the representative process to recombinantly prepare the chimeric enzymes. The chimeric enzyme was successively expressed in E. coli as a soluble protein, and was purified, as shown in SDSPAGE analyses.

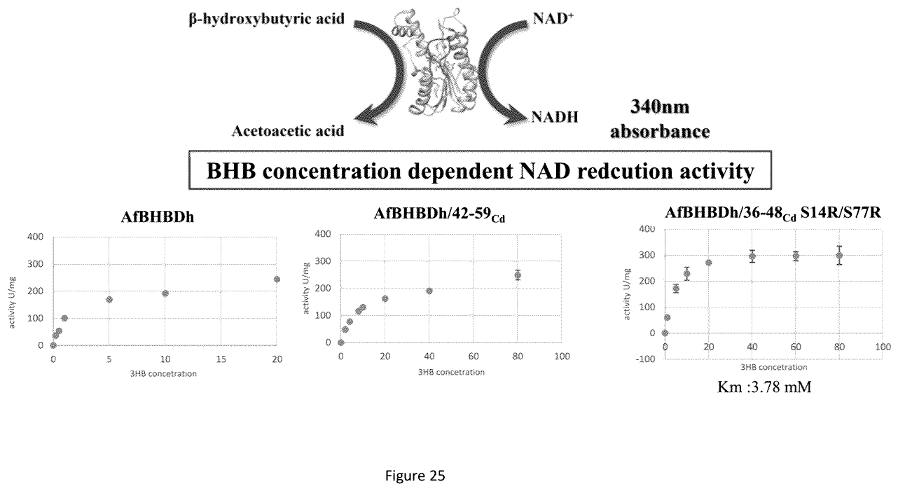

FIG. 25 shows the NAD dependent BHB dehydrogenase activities of AfBHBDh, and two chimeric enzymes, AfBHBDh 36-48_{Cd} and AfBHBDh 36-48_{Cd} S14R/S77R. Compared with the NAD dependent BHB dehydrogenase activity of AfBHBDh, the two chimeric enzymes, AfBHBDh 36-48_{Cd} and AfBHBDh 36-48_{Cd} S14R/S77R showed almost similar enzyme activity, revealing that the insertion of the 12 amino acid loop region of carveol dehydrogenase derived from Mycobacterium tuberculosis, with or without two amino acid substitutions (Ser14Arg/Ser71Arg) in AfBHBDh did not have any negative impact in the catalytic activity of AfBHBDh. Results are summarized in Table 2, below:

TABLE 2
NAD dependent BHB dehydrogenase activities of AfBHBDh, and two
chimeric enzymes, AfBHBDh 36-48_{Cd} and AfBHBDh 36-48_{Cd} S14R/S77R

| | Km(mM) | Vmax(U/mg) |
|---|---|---|
| AfBHBDh | 1.13 | 223 |
| AfBHBDh 36-48_{Cd} | 7.81 | 234 |
| AfBHBDh 36-48_{Cd} S14R/S77R | 3.78 | 315 |

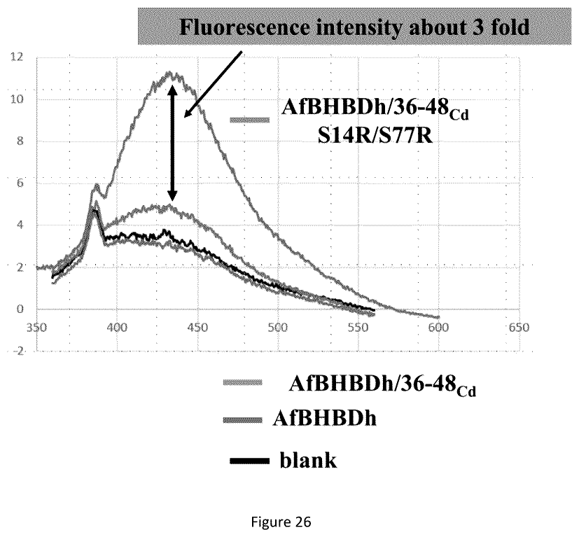

FIG. 26 shows the NADH dependent fluorescence observed from AfBHBDh, and two chimeric enzymes, AfBHBDh 36-48_{Cd} and AfBHBDh 36-48_{Cd} S14R/S77R. 200 μL of 1 mg/mL solution was prepared for each enzyme. The parameters of the fluorescence measurements were as follows:
Excitation spectra: Ex.: 250-400 nm, Em: 430 nm
Emission spectra: Ex.: 334 nm, Em: 360-560 nm

Compared with NADH dependent fluorescence of AfBHBDh, the two chimeric enzymes, AfBHBDh 36-48_{Cd} and AfBHBDh 36-48_{Cd} S14R/S77R showed 1.5 to 3 fold higher fluorescence intensity. These results revealed that the two chimeric enzymes bind with NAD and are therefore nicotinoproteins, which were created by inserting a 12 amino acid loop region of carveol dehydrogenase derived from Mycobacterium tuberculosis, with or without two amino acid substitutions (Ser14Arg/Ser71Arg).

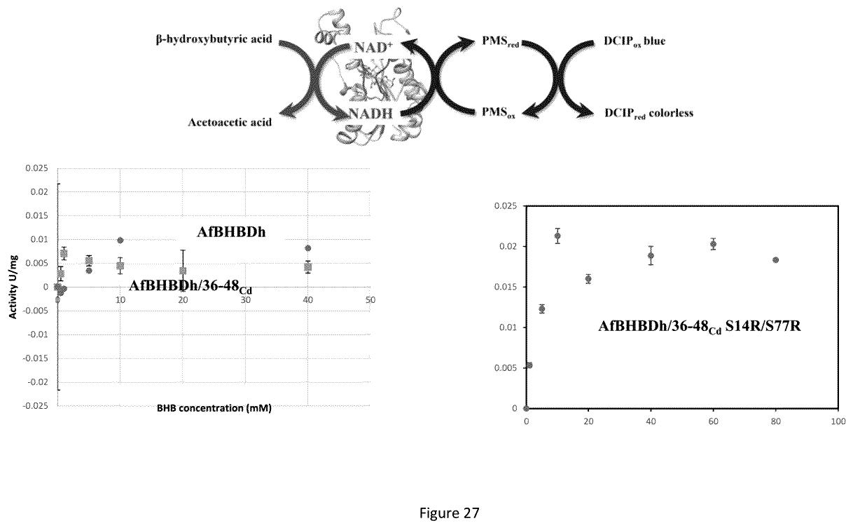

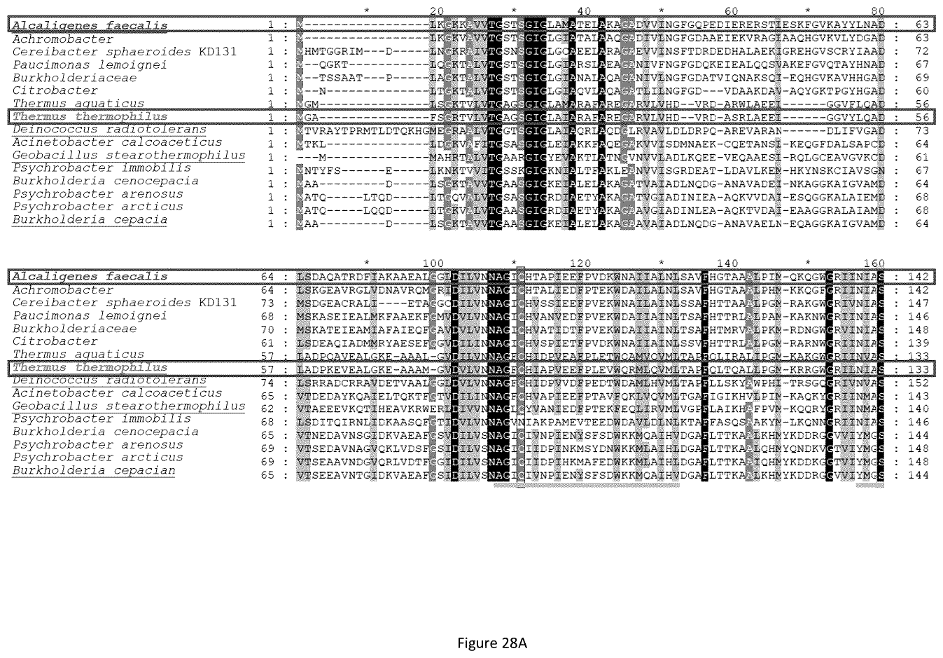

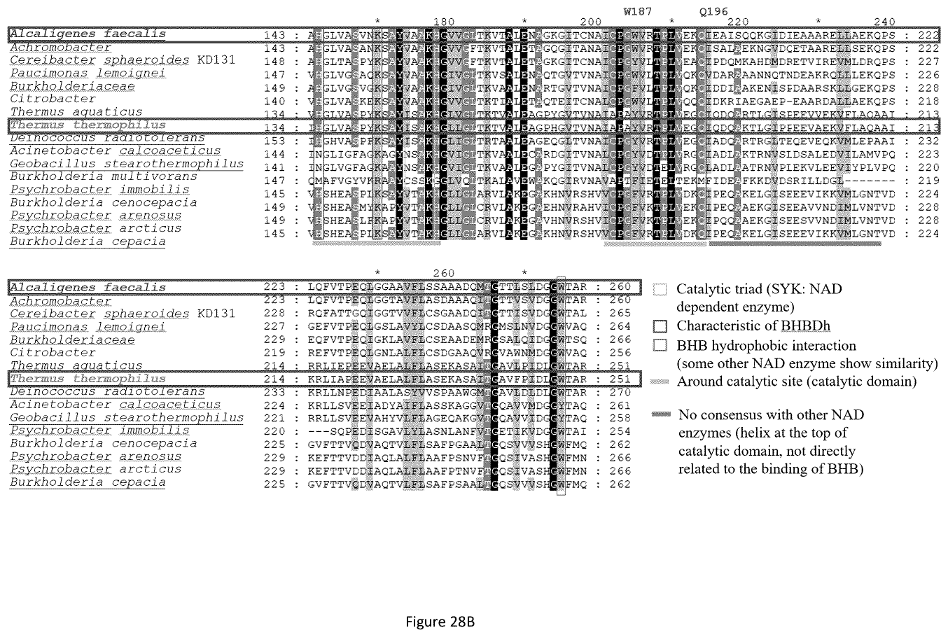

The chimeric enzymes, AfBHBDh 36-48_{Cd} and AfBHBDh 36-48_{Cd} S14R/S77R show dye-mediated BHB dehydrogenase activities in the absence of NAD⁺. FIG. 27 shows the dye-mediated dependent BHB dehydrogenase activities of AfBHBDh, and two chimeric enzymes, AfBHBDh 36-48_{Cd} and AfBHBDh 36-48_{Cd} S14R/S77R, in the absence of NAD⁺ but in the presence of PMC as the primary electron acceptor and DCIP as a color indicating dye. Compared with dye-mediated BHB dehydrogenase activity of AfBHBDh, the two chimeric enzymes, AfBHBDh 36-48_{Cd} and AfBHBDh 36-48_{Cd} S14R/S77R showed higher dye-mediated BHB dehydrogenase activities. Especially, AfBHBDh 36-48_{Cd} S14R/S77R, showed 2-3 fold higher dye-mediated dehydrogenase activity. These results indicated that by constructing a chimeric BHBDh harboring a 12 amino acid loop region of carveol dehydrogenase derived from Mycobacterium tuberculosis, with or without two amino acid substitutions (Ser14Arg/Ser71Arg), BHBDh can be converted into nicotinoprotein BHBDh.

Mutation Toward TtHBDh for the Recovery of Enzyme Activity

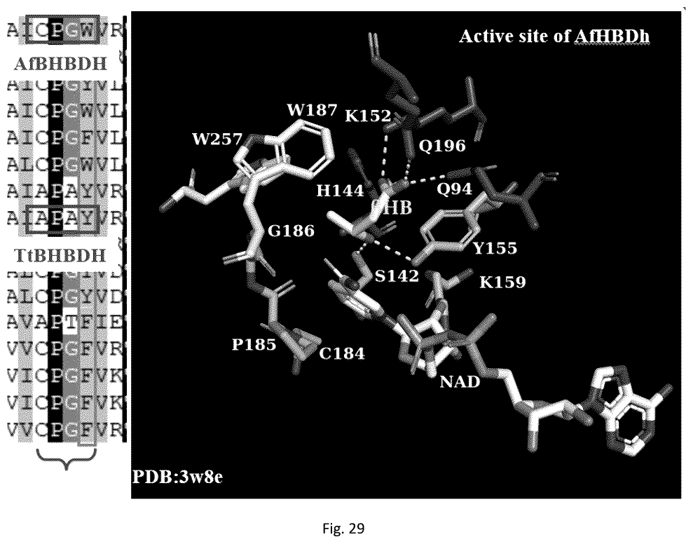

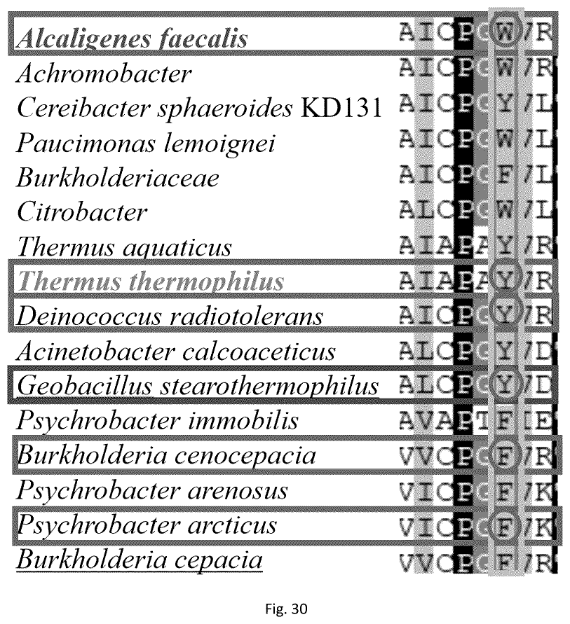

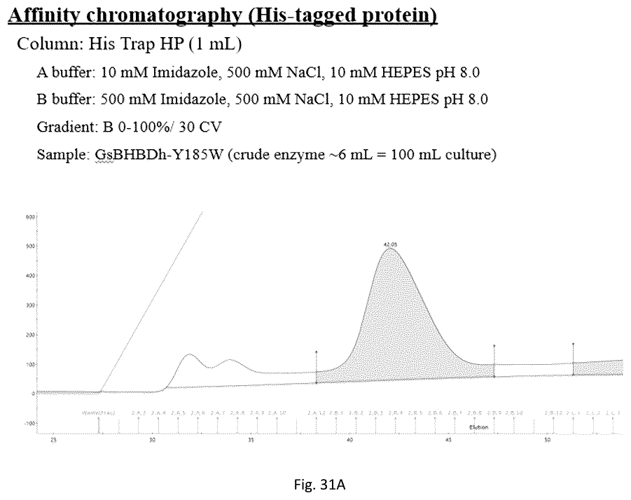

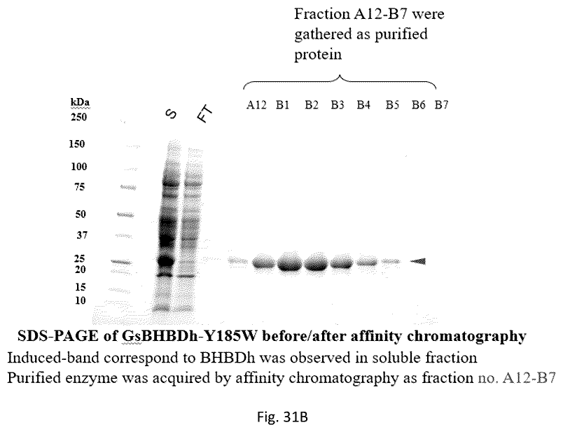

FIG. 29, on the left side, depicts the alignment of amino acid residues corresponding to positions 182-189 of AfBHBDh. Particularly highlighted are the differences between positions 184-187 of AfBHBDh (C184-P185-G186-W187) and correlated positions 175-178 of TtBHBDh (A175-P176-A177-Y178). A mutation of AfBHBDh (C184A-P185-G186A-W187Y) to match TtBHBDh's amino acids at these positions was performed, for the recovery of enzyme activity. The result of this mutation was an approximately 3-fold improvement in activity. Similarly, TtBHBDh (A175C-P176-A177G-Y178W) was mutated to match AfBHBDh's residues at these positions. However, this resulted in a drastic decrease in activity. It was understood from these results that this region has a significant effect on the enzyme's activity. Looking at the structure on the right side of FIG. 29, depicting the active site of AfBHBDh, it can be seen that the residues corresponding to these positions (184-187) are involved in the active site.

FIG. 30 depicts the alignment of the same segment of sequences, for numerous BHBDh enzymes. Residues corresponding to position 187 in AfBHBDh are circled. To further test the ability of mutations in this region to affect enzyme activity, a GsBHBDh Y185W mutant was created. Position 185 in GsBHBDh corresponds to position 187 in AfBHBDh.

FIG. 31A depicts the affinity chromatography scheme used to purify the His-tagged mutant protein. The column used was a His Trap HP (1 ML). The buffers used were an A buffer of 10 mM Imidazole, 500 mM NaCl, 10 mM HEPES at pH 8.0, and a B buffer of 500 mM Imidazole, 500 mM NaCl, 10 mM HEPES at pH 8.0, at a gradient of 0-100% B/30 CV. About 6 mL of crude enzyme was purified, from 100 mL of culture. FIG. 31B depicts the SDS-PAGE results of GsBHBDh-Y185W before and after affinity chromatography. A band corresponding to BHBDh was observed in the soluble fraction. Purified enzyme was acquired by affinity chromatography as fraction no. A12-B7.

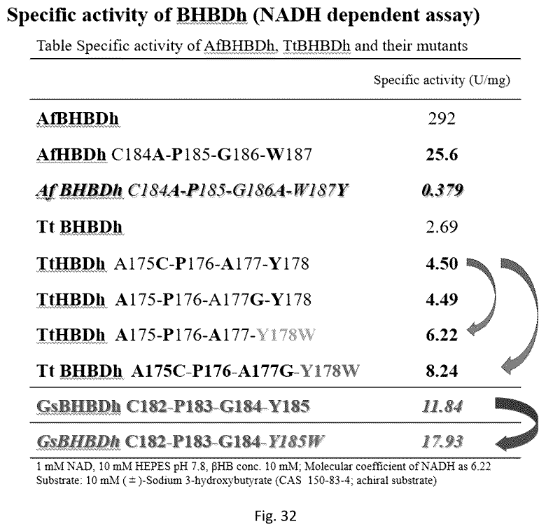

FIG. 32 depicts the specific activities (U/mg) of AfBHBDh wild type, TtBHBDh wild type, and certain AfBHBDh, TtBHBDh, and GsBHBDh mutants. The enzymes were tested in solutions comprising 1 mM NAD and 10 mM HEPES at pH 7.8, with a βHB concentration of 10 mM.

Notably, the Y185W mutation on GsBHBDh, which made its residues at positions 182-185 (CPGW) identical to those at the corresponding positions 184-187 in AfBHBDh, resulted in an approximately 50% increase in activity.

### CONCLUSION

Thermostable BHBDhs which overcome the limitations of the stability of current commercially available BHBDhs, which are not sufficiently thermostable for use in continuous monitoring systems. Considering the invented thermostable enzymes are not inactivated at 45° C., or even 65° C. or 75° C., these thermostable BHBDhs are much stable than the commercially available ones under dried conditions, and also during continuous operation in the solution at around 37° C. for 1-2 weeks, which is necessary for CKM.

A strategy and construction of nicotinoprotein BHBDhs is also disclosed. These nicotinoprotein BHBDhs bind to NAD in the molecule, like the currently utilized enzyme for continuous glucose monitoring, glucose oxidase, which harbors its redox cofactor (flavin adenine dinucleotide; FAD) tightly bound in its enzyme molecule. Therefore, it is no longer necessary to add NAD⁺ in the reaction solution.

In summary, these inventions solve the current biggest challenge in the limited availability of unstable BHBDh for BHB monitoring, and also requirement of the addition of NAD in the solution. These inventions will realize continuous BHB monitoring (continuous ketone monitoring; CKM). These inventions can be utilized jointly in a single engineered BHBDh, so as to create a thermostable nicotinoprotein BHBDh having a NAD bound tightly thereto.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims.

Although specific terms are employed herein, they are used in a generic and descriptive sense only and not for purposes of limitation. Each embodiment disclosed herein is contemplated as being applicable to each of the other disclosed embodiments. All combinations and sub-combinations of the various elements described herein are within the scope of the embodiments.

### LIST OF CITED REFERENCES

Alva, S. et al., Feasibility of Continuous Ketone Monitoring in Subcutaneous Tissue using a Ketone Sensor. J Diabetes Sci. Technol. 15, 768-774 (2021).
Batchelor, M. et al., Amperometric assay for the ketone body 3-hydroxybutyrate. Anal. Chim. Acta 221, 289-294 (1989).
Dhatariya, K., Blood ketones: Measurement, interpretation, limitations, and utility in the management of diabetic ketoacidosis. Rev. Diabet. Stud. 13, 217-225 (2016).
Dhatariya, K. et al., Diabetic ketoacidosis. Nat. Rev. Dis. Primers 6, 1-20 (2020).
Diabetes.co.uk., Ketone testing. Diabetes Digital Media (2019). https://www.diabetes.co.uk/diabetes_care/testing-for-ketones.html
European Medicines Agency (EMA), EMA confirms recommendations to minimise ketoacidosis risk with SGLT2 inhibitors for diabetes. 1-3 (2016).
Fang, L. et al. An electrochemical biosensor of the ketone 3-β-hydroxybutyrate for potential diabetic patient management. Sens. Actuators B Chem. 129, 818-825 (2008).
U.S. Food and Drug Administration (FDA), FDA warns that SGLT2 inhibitors for diabetes may result in a serious condition of too much acid in the blood. 1-4 (2015).
Forrow, N. et al., Development of a commercial amperometric biosensor electrode for the ketone d-3-hydroxybutyrate. Biosens. Bioelectron. 20, 1617-1625 (2005).
Kitabchi, A. et al., Hyperglycemic Crises in Adult Patients With Diabetes. Diabetes Care 32, 1335-1343 (2009).
Mitchell, G. et al., Medical aspects of ketone body metabolism. Clin. Invest. Med. 18, 193-216 (1995).
Laffel, L., Ketone bodies: a review of physiology, pathophysiology and application of monitoring to diabetes. Diabetes Metab. Res. Rev. 15, 412-426 (1999).
Lee, I. et al., Continuous glucose monitoring systems-current status and future perspectives of the flagship technologies in biosensor research. Biosens. Bioelectron. 181, 113054 (2021).
Li, G. et al., A new handheld biosensor for monitoring blood ketones. Sens. Actuators B Chem. 109, 285-290 (2005).
Marrazza, G. et al., NADH electrochemical sensor for the enzymatic determination of L- and D-lactate and 3-hydroxybutyrate using a flow injection analysis. Electroanalysis 6, 221-226 (1994).
Martinez-Garcia, G. et al., An electrochemical enzyme biosensor for 3-hydroxybutyrate detection using screen-printed electrodes modified by reduced graphene oxide and thionine. Biosensors 7, 50 (2017).
Mascini, M. et al., In-line determination of metabolites and milk components with electrochemical biosensors. Anal. Chim. Acta 213, 101-111 (1988).
McNeil, C. et al., Amperometric biosensor for rapid measurement of 3-hydroxybutyrate in undiluted whole blood and plasma. Anal. Chim. Acta 237, 99-105 (1990).
Palleschi, G. et al., Amperometric probe for 3-hydroxybutyrate with immobilized 3-hydroxybutyrate dehydrogenase. Anal. Chim. Acta 209, 223-230 (1988).
Peters, A. et al., Diabetic Ketoacidosis With Canagliflozin, a Sodium-Glucose Cotransporter 2 Inhibitor, in Patients With Type 1 Diabetes. Diabetes Care 39, 532-538 (2016).
Plüddemann A. et al., Point-of-care blood test for ketones in patients with diabetes: primary care diagnostic technology update. Br. J Gen. Pract. 61, 530-531 (2011).
Rosenstock, J. and Ferrannini, E., Euglycemic diabetic ketoacidosis: A predictable, detectable, and preventable safety concern with sglt2 inhibitors. Diabetes Care 38, 1638-1642 (2015).
Teymourian, H. et al., Microneedle-Based Detection of Ketone Bodies along with Glucose and Lactate: Toward Real-Time Continuous Interstitial Fluid Monitoring of Diabetic Ketosis and Ketoacidosis. Anal. Chem. 92, 2291-2300 (2020).
Uno, S. et al., Enzymatic method for determining ketone body ratio in arterial blood. Clin. Chem. 41, 1745-1750 (1995).
Veerapandian, M. et al., Ruthenium dye sensitized graphene oxide electrode for on-farm rapid detection of beta-hydroxybutyrate. Sens. Actuators B Chem. 228, 180-184 (2016).
Vojinovic, V. et al., Real-time bioprocess monitoring: part I: in situ sensors. Sens. Actuators B Chem. 114, 1083-1091 (2006).
Wallace, T. and Matthews, D., Recent advances in the monitoring and management of diabetic ketoacidosis. QJM: An International Journal of Medicine 97, 773-780 (2004).

1. An engineered β-hydroxybutyrate dehydrogenase (BHBDh) with increased thermal stability as compared to the wild-type.

2. The engineered BHBDh of claim 1, comprising a sequence having at least 90% sequence identity to any one of SEQ ID NOs: 1-7 or 12-20, provided at least one of the amino acids at a position in said sequence corresponding to positions 165-188 is different from the amino acid occupying the corresponding position according to SEQ ID NO: 3.

3. The engineered BHBDh of claim 2, comprising a modification at one or more amino acid positions selected from:
(a) a position corresponding to position 175 of the amino acid sequence set forth in SEQ ID NO: 3,
(b) a position corresponding to position 177 of the amino acid sequence set forth in SEQ ID NO: 3, and
(c) a position corresponding to position 178 of the amino acid sequence set forth in SEQ ID NO: 3.

4. The engineered BHBDh of claim 3, comprising a modification at one or more amino acid positions selected from:
(a) a position corresponding to position 175 of the amino acid sequence set forth in SEQ ID NO: 3, wherein the modification includes a substitution of the wild-type amino acid residue with an amino acid residue Cys;
(b) a position corresponding to position 177 of the amino acid sequence set forth in SEQ ID NO: 3, wherein the modification includes a substitution of the wild-type amino acid residue with an amino acid residue Gly; and
(c) a position corresponding to position 178 of the amino acid sequence set forth in SEQ ID NO: 3, wherein the modification includes a substitution of the wild-type amino acid residue with an amino acid residue Trp.

5. (canceled)

6. The engineered BHBDh of any of the preceding claims, comprising a sequence having at least 90% sequence identity to any one of SEQ ID NOs: 3-5.

7-8. (canceled)

9. An engineered β-hydroxybutyrate dehydrogenase (BHBDh) having a NAD-binding sequence inserted therein.

10. The engineered BHBDh of claim 9, wherein the BHBDh has a sequence having at least 90% sequence identity to any one of SEQ ID NOs: 1-7 or 12-20.

11. (canceled)

12. The engineered BHBDh of claim 10, wherein at least one of the amino acids at a position in said sequence corresponding to positions 4-24, and at least one of the amino acids at a position in said sequence corresponding to positions 61-81, are different from the amino acids occupying the corresponding positions according to SEQ ID NO: 1.

13. The engineered BHBDh of claim 12, comprising a modification at one or more amino acid positions selected from:
(a) a position corresponding to position 14 of the amino acid sequence set forth in SEQ ID NO: 1; and
(b) a position corresponding to position 71 of the amino acid sequence set forth in SEQ ID NO: 1.

14. The engineered BHBDh of claim 13, comprising a modification at one or more amino acid positions selected from:
(a) a position corresponding to position 14 of the amino acid sequence set forth in SEQ ID NO: 1, wherein the modification includes a substitution of the wild-type amino acid residue with an amino acid residue Arg; and
(b) a position corresponding to position 71 of the amino acid sequence set forth in SEQ ID NO: 1, wherein the modification includes a substitution of the wild-type amino acid residue with an amino acid residue Arg.

15. The engineered BHBDh of claim 10, wherein at least one of the amino acids at a position in said sequence corresponding to positions 165-188 is different from the amino acid occupying the corresponding position according to SEQ ID NO: 3.

16. The engineered BHBDh of claim 15, comprising a modification at one or more amino acid positions selected from:
(a) a position corresponding to position 175 of the amino acid sequence set forth in SEQ ID NO: 3,
(b) a position corresponding to position 177 of the amino acid sequence set forth in SEQ ID NO: 3, and
(c) a position corresponding to position 178 of the amino acid sequence set forth in SEQ ID NO: 3.

17. The engineered BHBDh of claim 16, comprising a modification at one or more amino acid positions selected from:
(a) a position corresponding to position 175 of the amino acid sequence set forth in SEQ ID NO: 3, wherein the modification includes a substitution of the wild-type amino acid residue with an amino acid residue Cys;
(b) a position corresponding to position 177 of the amino acid sequence set forth in SEQ ID NO: 3, wherein the modification includes a substitution of the wild-type amino acid residue with an amino acid residue Gly; and
(c) a position corresponding to position 178 of the amino acid sequence set forth in SEQ ID NO: 3, wherein the modification includes a substitution of the wild-type amino acid residue with an amino acid residue Trp.

18-31. (canceled)

32. The engineered BHBDh of claim 9, having a nicotinamide adenine dinucleotide (NAD) bound thereto.

33. An expression vector comprising a nucleic acid sequence encoding the engineered BHBDh of claim 1.

34. A method of producing the engineered BHBDh of claim 1, the method comprising the steps of
i) culturing a host cell transfected with a vector comprising a nucleic acid sequence encoding the engineered BHBDh under conditions suitable for expression of the polypeptide from the host cell, and
ii) recovering the engineered BHBDh.

35. A method of assaying β-hydroxybutyrate (BHB) in a sample, the method comprising the steps of:
contacting the sample with the engineered BHBDh of claim 1; and measuring an amount of the BHB oxidized by the engineered BHBDh.

36. The method of claim 35, wherein the measurement is a continuous measurement.

37. A device for assaying BHB in a sample, the device comprising: the engineered BHBDh of claim 1; and an electron mediator.

38. (canceled)

39. An enzyme electrode comprising the engineered BHBDh of claim 1 immobilized on an electrode.

40. An enzyme sensor for assaying BHB comprising the enzyme electrode of claim 39 as a working electrode.


SEQUENCE LISTING (20 sequences)

SEQ ID NO: 1 - protein, 260 aa, Alcaligenes faecalis [sequence omitted]

SEQ ID NO: 2 - protein, 251 aa, Thermus aquaticus
MGMLSGKTVLVTGAGSGIGLAMARAFAREGARVLVHDVRDARWLAEELGGVFLQADLADPQAVEALGKEAAALGVDILVNNAGFQHIDPVEAFPLETWQAMVQVMLTAPFQLIRALIPGMKAKGWGRIINVASVHGLVASPYKSAYISAKHGLVGLTKTVALEAGPYGVTVNAIAPAYVRTPLVEGQIQDQARTLGISPEEVVEKVFLAQAAIRRLIEPEEVAELALFLASEKASAITGAVLPIDLGWTAR

SEQ ID NO: 3 - protein, 251 aa, Thermus thermophilus
MGAFSGRTVLVTGAGSGIGLAIARAFAREGARVLVHDVRDASRLAEELGGVYLQADLADPKEVEALGKEAAAMGVDVLVNNAGFQHIAPVEEFPLEVWQRMLQVMLTAPFQLTQALLPGMKRRGWGRILNIASIHGLVASPYKSAYISAKHGLLGLTKTVALEAGPHGVTVNAIAPAYVRTPLVEGQIQDQAKTLGIPPEEVAEKVFLAQAAIKRLIAPEEVAELALFLASEKASAITGAVFPIDLGWTAR

SEQ ID NO: 4 - protein, 270 aa, deinococcus radiotolerans
MTVRAYTPRMTLDTQKHGMEGRAALVTGGTSGIGLAIAQRLAQDGLRVAVLDLDRPQAREVARANDLIFVGADLSRRADCRRAVDETVAALGGLDVLVNNAGFQHIDPVVDFPEDTWDAMLHVMLTAPFLLSKYAWPHLTRSGQGRIVNVASIHGHVASPFKSAYISAKHGLIGLTRTAALEAGEQGLTVNAICPGYVRTPLVEGQIADQARTRGLTEQEVEQKVMLEPAAIKRLLNPEDIAALASYVVSPAAWGMTGAVLDLDLGWTAR

SEQ ID NO: 5 - protein, 258 aa, Geobacillus stearothermophilus
MMAHRTALVTGAARGIGYEVAKTLATNGVNVVLADLKQEEVEQAAESLRQLGCEAVGVKCDVTAEEEVKQTIHEAVKRWERLDIVVNNAGLQYVANIEDFPTEKFEQLIRVMLVGPFLAIKHAFPVMKQQRYGRIINMASINGLVGFAGKAAYNSAKHGVIGLTKVAALEGAPYGITVNALCPGYVDTELVRGQLADLAATRNVPLEKVLEEVIYPLVPQRRLLSVEEVAHYVLFLAGEQAKGVTGQAVVIDGGYTAQ

SEQ ID NO: 6 - protein, 262 aa, Burkholderia cenocepacia
MAADLSGKTAVVTGAASGIGKEIALELAKAGATVAIADLNQDGANAVADEINKAGGKAIGVAMDVTNEDAVNSGIDKVAEAFGSVDILVSNAGIQIVNPIENYSFSDWKKMQAIHVDGAFLTTKAALKHMYKDDRGGVVIYMGSVHSHEASPLKSAYVTAKHGLLGLARVLAKEGAKHNVRSHVVCPGFVRTPLVDKQIPEQAKELGISEEEVIKKVMLGNTVDGVFTTVQDVAQTVLFLSAFPGAALTGQSVVVSHGWFMQ

SEQ ID NO: 7 - protein, 262 aa, Burkholderia cepacia
MAADLSGKTAVVTGAASGIGKEIALELAKAGAAVAIADLNQDGANAVAELNEQAGGKAIGVAMDVTSEEAVNTGIDKVAEAFGSIDILVSNAGIQIVNPIENYSFSDWKKMQAIHVDGAFLTTKAALKHMYKDDRGGVVIYMGSVHSHEASPLKSAYVTAKHGLLGLARVLAKEGAKHNVRSHVVCPGFVRTPLVDKQIPEQAKELGISEEEVIKKVMLGNTVDGVFTTVQDVAQTVLFLSAFPSAALTGQSVVVSHGWFMQ

SEQ ID NO: 8 - protein, 272 aa, synthetic construct
MLKGKKAVVTGSTSGIGLAMATELAKAGADVVINGICAPVSASVTYAPGQPEDIERERSTLESKFGVKAYYLNADLSDAQATRDFIAKAAEALGGLDILVNNAGIQHTAPIEEFPVDKWNAIIALNLSAVFHGTAAALPIMQKQGWGRIINIASAHGLVASVNKSAYVAAKHGVVGLTKVTALENAGKGITCNAICPGWVRTPLVEKQIEAISQQKGIDIEAAARELLAEKQPSLQFVTPEQLGGAAVFLSSAAADQMTGTTLSLDGGWTAR

SEQ ID NO: 9 - protein, 272 aa, synthetic construct
MLKGKKAVVTGSTRGIGLAMATELAKAGADVVINGICAPVSASVTYAPGQPEDIERERSTLESKFGVKAYYLNADLRDAQATRDFIAKAAEALGGLDILVNNAGIQHTAPIEEFPVDKWNAIIALNLSAVFHGTAAALPIMQKQGWGRIINIASAHGLVASVNKSAYVAAKHGVVGLTKVTALENAGKGITCNAICPGWVRTPLVEKQIEAISQQKGIDIEAAARELLAEKQPSLQFVTPEQLGGAAVFLSSAAADQMTGTTLSLDGGWTAR

SEQ ID NO: 10 - protein, 12 aa, Mycobacterium tuberculosis
ICAPVSASVTYA

SEQ ID NO: 11 - protein, 20 aa, synthetic construct
MGSSHHHHHHSSGLVPRGSH

SEQ ID NO: 12 - protein, 260 aa, Achromobacter sp.
MLKGKVAVVTGSTSGIGLGIATALAAQGADIVLNGFGDAAEIEKVRAGLAAQHGVKVLYDGADLSKGEAVRGLVDNAVRQMGRIDILVNNAGIQHTALIEDFPTEKWDAILALNLSAVFHGTAAALPHMKKQGFGRIINIASAHGLVASANKSAYVAAKHGVVGFTKVTALETAGQGITANAICPGWVRTPLVEKQISALAEKNGVDQETAARELLSEKQPSLQFVTPEQLGGTAVFLASDAAAQITGTTVSVDGGWTAR

SEQ ID NO: 13 - protein, 265 aa, cereibacter spaeroides
MHMTGGRIMDLNGKRAIVTGSNSGIGLGCAEELARAGAEVVINSFTDRDEDHALAEKIGREHGVSCRYIAADMSDGEACRALIETAGGCDILVNNAGIQHVSSIEEFPVEKWNAILAINLSSAFHTTAAALPGMRAKGWGRIVNIASAHGLTASPYKSAYVAAKHGVVGFTKVTALETAGKGITCNAICPGYVLTPLVEAQIPDQMKAHDMDRETVIREVMLDRQPSRQFATTGQIGGTVVFLCSGAADQITGTTISVDGGWTAL

SEQ ID NO: 14 - protein, 264 aa, Paucimonas lemoignei
MQGKTLQGKTALVTGSTSGIGLGIARSLAEAGANIVFNGFGDQKEIEALQQSVAKEFGVQTAYHNADMSKASEIEALMKFAAEKFGMVDVLVNNAGIQHVANVEDFPVEKWDAIIAINLTSAFHTTRLALPAMKAKNWGRIINIASVHGLVGSAQKSAYVAAKHGIVGLTKVSALENAQTGVTVNAICPGWVLTPLVQKQVDARAAANNQTNDEAKRQLLLEKQPSGEFVTPEQLGSLAVYLCSDAASQMRGMSLNVDGGWVAQ

SEQ ID NO: 15 - protein, 266 aa, Burkholderia sp.
MTSSAATPLAGKTALVTGSTSGIGLGIANALAQAGANIVLNGFGDATVIQNAKSQIEQHGVKAVHHGADMSKATEIEAMIAFAIEQFGAVDVLVNNAGIQHVATIDTFPVEKWDAIIAINLTSAFHTMRVALPKMRDNGWGRVINIASAHGLVGSVGKSAYVAAKHGIVGLTKVAALENARTGVTVNAICPGFVLTPLVQKQIDDIAAKENISPDAARSKLLGEKQPSEQFVTPEQIGKLAVFLCSEAADEMRGSALQIDGGWTSQ

SEQ ID NO: 16 - protein, 256 aa, Citrobacter sp.
MNLTGKTALVTGSTSGIGLGIAQVLAQAGATLILNGFGDVDAAKDAVAQYGKTPGYHGADLSDEAQIADMMRYAESEFGGVDILINNAGIQHVSPIETFPVDKWNAIIAINLSSVFHTTRLALPGMRARNWGRIINIASVHGLVASKEKSAYVAAKHGVVGLTKTIALETAQTEITCNALCPGWVLTPLVQQQIDKRIAEGAEPEAARDALLAEKQPSREFVTPEQLGNLALFLCSDGAAQVRGVAWNMDGGWVAQ

SEQ ID NO: 17 - protein, 261 aa, Acinetobacter calcoaceticus
MTKLLDGKVAFITGSASGIGLEIAKKFAQEGAKVVISDMNAEKCQETANSLKEQGFDALSAPCDVTDEDAYKQAIELTQKTFGTVDILINNAGFQHVAPIEEFPTAVFQKLVQVMLTGAFIGIKHVLPIMKAQKYGRIINMASINGLIGFAGKAGYNSAKHGVIGLTKVAALECARDGITVNALCPGYVDTPLVRGQIADLAKTRNVSLDSALEDVILAMVPQKRLLSVEEIADYAIFLASSKAGGVTGQAVVMDGGYTAQ

SEQ ID NO: 18 - protein, 254 aa, Psychrobacter immobilis
MNTYFSELKNKTVVITGSSKGIGKNIALTFAKLEANVVISGRDEATLDAVLKEMHKYNSKCIAVSGNLSDITQIRNLIDKAASQFGTIDVLVNNAGVNIAKPAMEVTEEDWDAVLDLNLKTAFFASQSAAKYMLKQNNGRIINIASQMAFVGYVKRAAYCSSKGGLVQLTKALAVEWAKQGIRVNAVAPTFIETELTEKMFIDEAFKKDVDSRILLDGLSQPEDISGAVLYLASNLANFVTGETIKVDGGWTAI

SEQ ID NO: 19 - protein, 266 aa, Psychrobacter arenosus
MATQLTQDLTGQVALVTGSASGIGRDIAETYAKAGATVGIADINIEAAQKVVDAIESQGGKALAIEMDVTSEDAVNAGVQKLVDSFGSIDILVSNAGIQIIDPINKMSYDNWKKMLAIHLDGAFLTTKAALQHMYQNDKVGTVIYMGSVHSHEASMYKAPYVTAKHGLLGLCRVLAKEGAEHNVRAHVICPGFVKTPLVEKQIPEQAAEKGISEEAVVNDIMLVNTVDKEFTTVDDIAQLALFLAAFPSNVFTGQSIVASHGWFMN

SEQ ID NO: 20 - protein, 266 aa, Psychrobacter arcticus
MATQLQQDLTGKVALVTGAASGIGRDIAETYAKAGAAVGIADINLEAAQKTVDAIEAAGGRALAIAMDVTSEAAVNDGVQRLVDTFGGIDILVSNAGIQIIDPIHKMAFEDWKKMLAIHLDGAFLTTKAAIQHMYKDDKGGTVIYMGSVHSHEASLFKAPYVTAKHGLLGLCRVLAKEGAVHNVRSHVICPGFVKTPLVEKQIPQQAAEKGISEESVVNDIMLVNTVDKEFTTVDDIAQLALFLAAFPTNVFTGQSIVASHGWFMN